(19) United States
(12) Patent Application Publication
(10) Pub. No.: US 2024/0082167 A1
(43) Pub. Date: Mar. 14, 2024

(54) SYNTHETIC ARTIFICIAL STEM CELLS (SASC)

(71) Applicant: UNIVERSITY OF CONNECTICUT, Farmington, CT (US)

(72) Inventors: Cato LAURENCIN, Farmington, CT (US); Shiv S. SHAH, Farmington, CT (US); Lakshmi NAIR, Farmington, CT (US); Ho-man KAN, Farmington, CT (US)

(21) Appl. No.: 18/262,443

(22) PCT Filed: Jan. 21, 2022
(86) PCT No.: PCT/US2022/013244

§ 371 Date: Jul. 21, 2023

Related U.S. Application Data
(60) Provisional application No. 63/140,537, filed on Jan. 22, 2021.

(51) Int. Cl. A61K 9/50; A61K 9/19; A61K 38/18; A61K 45/06; A61P 19/02

(52) U.S. Cl. CPC A61K 9/5031; A61K 9/19; A61K 38/1825; A61K 38/1841; A61K 45/06; A61P 19/02

(57) ABSTRACT

This disclosure relates to an engineered a novel synthetic artificial stem cell (SASC) system which mimics the paracrine effect of the stem cell secretome and provides tailorability of the composition for targeted tissue regeneration. This novel SASC system demonstrates the feasibility of developing a completely synthetic, tailorable stem cell secretome which reinforces the possibility of developing a new therapeutic strategy that provides better control over targeted tissue engineering applications. Disclosed herein are biodegradable polymeric microspheres that have been used to encapsulate secretome active agents. These microspheres mimic or have the potential to result in a greater therapeutic paracrine effect compared to stem cells, without the added risk of immunological response or the extra pro-inflammatory, antagonistic or inert factors/cytokines. The compositions may be personalized to any disease and/or individual, therefore, adding a much needed element to therapeutics.

### CROSS REFERENCE TO RELATED APPLICATIONS

This application claims priority to U.S. Provisional Patent Application Ser. No. 63/140,537, filed Jan. 22, 2021, which is incorporated by referenced herein in its entirety.

### FIELD OF THE DISCLOSURE

The secretome is a group of growth factors and cytokines that are released by cells into their microenvironment. These secreted growth factors have various therapeutic effects such as anti-inflammation, anti-apoptosis and angiogenesis, though there are some factors in the secretome which may have no therapeutic benefit or have an antagonistic effect on the regenerating tissue.

This disclosure presents a Synthetic Artificial Stem Cell (SASC) system: a versatile therapy which provides the ability to tailor paracrine responses of different cells and provide a more potent regenerative effect for targeted tissues. The system is a tailorable therapy that mimics the paracrine function of a stem cell. As proof of principle, upon challenging the SASC system against an osteoarthritis (OA) model, this disclosure demonstrates that the factors combined tailored for chondrogenesis have a potent anti-inflammatory and chondroprotective effect. This disclosure also demonstrates the in vivo capacity of SASC to attenuate proteoglycan depletion in the cartilage extracellular matrix while also improving biomechanical properties of the resulting cartilage. This is a first demonstration of many applications of the SASC system which provides a promising step toward the clinical translation of a minimally immunogenic stem cell with many commercial advantages over its biological counterpart.

### BACKGROUND

Stem cell therapy focuses on the delivery of cells to facilitate tissue repair and regeneration by a combination of anti-inflammatory, immunomodulatory properties and multipotent differentiation capacity. However, in order to be used in a clinical setting, they must be isolated from human tissue and require constant growth and passage in in vitro culture environments. In addition, studies have reported that cells are at risk for undergoing spontaneous alterations in behavior and properties while being cultured⁹. Contamination is also a risk due to improper technique or non-sterile conditions. Other limitations and challenges that should be considered when developing stem cell therapies include immune compatibility and rejection reactions, formation of malignant tumors due to uncontrolled proliferation and transmission of infectious processes^{10,11}.

### SUMMARY

Disclosed herein are biodegradable polymeric microspheres that have been used to encapsulate secretome active agents. These microspheres mimic or have the potential to result in a greater therapeutic paracrine effect compared to stem cells, without the added risk of immunological response or the extra pro-inflammatory, antagonistic or inert factors/cytokines. The composition may be personalized to any disease and/or individual, therefore, adding a much needed element to therapeutics.

In one aspect, this disclosure provides a composition comprising one or more populations of microspheres, wherein each of the one or more populations of microspheres comprises at least one active agent. In some embodiments, the microspheres comprise a polymeric waxy or other protective material such as a natural, a semi-synthetic, or a synthetic polymer. In certain embodiments, the microspheres comprise a biocompatible polymer selected from: Poly(lactic-co-glycolic acid) (PLGA), Poly(lactic acid) (PLA), Poly(ϵ-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof.

In some embodiments, the microspheres have a diameter ranging from about 1 μm to about 100 μm. In some embodiments, the microspheres have a diameter ranging from about 10 μm to about 20 μm.

In some embodiments, the at least one active agent comprises one or more active agents selected from: growth factors, chemokines, cytokines, CD antigens, neurotrophins, and microRNAs (miRNAs).

In some embodiments, the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMPS), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGFS), Fibroblast growth factor 6 (FGF6), Fibroblast growth factor 7(FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDFS), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth Factor, Kruppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1), Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOX5, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-β), Transforming Growth Factor-β2 (TGF-(β2), Transforming Growth Factor-β3 (TGF-(β3), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor -D (VEGF-D).

In some embodiments, the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCL5), Eotaxin (CCL11,24,26), Exodus-2 (CCL21), Fractalkine (CX3CL1), GRO (CXCL1,2,3), HCC-1 (CCL14), I-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCLS), SDF (CXCL12), TARC (CCL17), CCL14, CCL19, CCL20, CCL27, CXCL13, and Thymus Expressed Chemokine (CCL25).

In some embodiments, the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36γ, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, and Wingless-Type MMTV Integration Site Family.

In some embodiments, the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CD5, CD8B, CD5L, CD68, CD55, CD7, CD73, CD58, CD74, CD80, CD79, CD84, CD93, CD99, CD164, and CD40L.

In some embodiments, the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, and Pleiotrophin.

In some embodiments, the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

In an embodiment, the at least one active agent comprises VEGF, IGF-1, TGF-β, HGH, and FGF-18.

In another embodiment, the at least one active agent comprises IGF-1, TGF-β, HGH, and FGF-18.

In some embodiments, the composition comprises about 20% by weight FGF-18 microspheres, about 20% by weight TGF-(31 microspheres, about 20% by weight IGF-1 microspheres, about 20% by weight HGH microspheres, and about 20% by weight empty microspheres. In some embodiments, the composition comprises about 25% by weight FGF-18 microspheres, about 25% by weight TGF-(31 microspheres, about 25% by weight IGF-1 microspheres, and about 25% by weight HGH microspheres.

In some embodiments, the composition comprises about 1 ng to about 100 ng HGH, about 100 ng HGH, about 90 ng HGH, about 80 ng HGH, about 70 HGH, about 60 ng HGH, about 50 ng HGH, about 40 ng HGG, about 30 ng HGH, about 20 ng HGH, about 10 ng HGH, about 9.256 ng HGH, about 9 ng HGH, about 8 ng HGH, about 7 ng HGH, about 6 ng HGH, about 5 ng HGH, about 4 ng HGH, about 3 ng HGH, about 2 ng HGH, or about 1 ng HGH.

In some embodiments, the composition comprises about 1 ng to about 100 ng FGF-18, about 100 ng FGF-18, about 90 ng FGF-18, about 80 ng FGF-18, about 70 FGF-18, about 60 ng FGF-18, about 50 ng FGF-18, about 40 ng FGF-18, about 30 ng FGF-18, about 20 ng FGF-18, about 10 ng FGF-18, about 9.256 ng FGF-18, about 9 ng FGF-18, about 8 ng FGF-18, about 7 ng FGF-18, about 6 ng FGF-18, about 5 ng FGF-18, about 4 ng FGF-18, about 3 ng FGF-18, about 2 ng FGF-18, or about 1 ng FGF-18.

In some embodiments, the composition comprises about 1 ng to about 100 ng IGF-1, about 100 ng IGF-1, about 90 ng IGF-1, about 80 ng IGF-1, about 70 IGF-1, about 60 ng IGF-1, about 50 ng IGF-1, about 40 ng IGF-1, about 30 ng IGF-1, about 20 ng IGF-1, about 11.86 ng IGF-1, about 10 ng IGF-1, about 9 ng IGF-1, about 8 ng IGF-1, about 7 ng IGF-1, about 6 ng IGF-1, about 5 ng IGF-1, about 4 ng IGF-1, about 3 ng IGF-1, about 2 ng IGF-1, or about 1 ng IGF-1.

In some embodiments, the composition comprises about 1 ng to about 100 ng TGF-β1, about 100 ng TGF-β1, about 90 ng TGF-β1, about 80 ng TGF-β1, about 70 TGF-β1, about 60 ng TGF-β1, about 50 ng TGF-β1, about 40 ng TGF-β1, about 30 ng TGF-β1, about 20 ng TGF-β1, about 10 ng TGF-β1, about 9.256 ng TGF-β1, about 9 ng TGF-β1, about 8 ng TGF-β1, about 7 ng TGF-β1, about 6 ng TGF-β1, about 5.424 ng TGF-β1, about 5 ng TGF-β1, about 4 ng TGF-β1, about 3 ng TGF-β1, about 2 ng TGF-β1, or about 1 ng TGF-β1.

In some embodiments, wherein the at least one active agent comprises VEGF, TGF-β1, and BMP2. In some embodiments, the at least one active agent comprises Myostatin, IGF-1, and Growth differentiation factor 11. In some embodiments, the at least one active agent comprises Oct-4, SOX2, KLF4, and c-Myc.

In some embodiments, the biocompatible polymer comprises PLGA (Poly(lactic-co-glycolic acid)). In some embodiments, the Poly(lactic-co-glycolic acid) consists of an 85:15 Lactic Acid:Glycolic acid ratio in the polymer.

In some embodiments, the composition comprises two or more populations of microspheres and each population of microspheres comprises a single active agent.

In some embodiments, the composition comprises one population of microspheres and the population of microspheres comprises two or more active agents.

In some embodiments, the at least one active agent further comprises a carrier protein.

In some embodiments, the carrier protein is selected from bovine serum albumin, human serum albumin, equine serum albumin, goat serum albumin, porcine serum albumin, rat serum albumin, mouse serum albumin, chicken serum albumin, and chicken white albumin.

In some embodiments, the composition further comprises one or more delivery vehicles, diluents, excipients, pharmaceutical adjuvants, stimulants, and/or stabilizers.

In another aspect, this disclosure provides methods for treating a subject, comprising administering an effective dose of the composition as described and disclosed herein.

In some embodiments, the subject is suspected of having or has a degenerative disease. In some embodiments, the subject is suspected of having or has a disease selected from the group consisting of: Alzheimer's disease, spinal cord injury, muscular dystrophy, osteoporosis, and osteoarthritis, and the method serves to treat Alzheimer's disease, spinal cord injury, muscular dystrophy, osteoporosis, and osteoarthritis. In some embodiments, the subject has a spinal cord injury, muscular dystrophy, osteoporosis, and/or osteoarthritis.

In some embodiments, this disclosure provides a method for treating osteoarthritis (OA) in a subject, comprising administering an effective dose of the composition as disclosed herein. In some embodiments, this disclosure provides a method for repairing bone defects in a subject, comprising administering an effective dose of the composition as described herein. In some embodiments, this disclosure provides a method for attenuating skeletal muscle degeneration in a subject, comprising administering an effective dose of the composition as described herein. In some embodiments, this disclosure provides a method for reprogramming cells in a subject, comprising administering an effective dose of the composition as described herein.

In yet another aspect, this disclosure provides a method for preparing a composition comprising microspheres and at least one active agent, the method comprising:

(a) dissolving a polymer in a polar solvent and mixing to prepare a dissolved polymer;

(b) mixing the at least one active agent with a buffer and a carrier protein to prepare a diluted active agent;

(c) combining the dissolved polymer with the diluted active agent and mixing to prepare a primary emulsion;

(d) mixing the primary emulsion with an aqueous surfactant to make a secondary emulsion;

(e) mixing the secondary emulsion in a bulk aqueous solution comprising a salt, the aqueous surfactant, and an alcohol until the polar solvent is evaporated; and

(f) isolating the microspheres.

In some embodiments, the polymer is selected from: Poly(lactic-co-glycolic acid) (PLGA), Poly(lactic acid) (PLA), Poly(ϵ-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof In some embodiments, the biocompatible polymer comprises PLGA (Poly(lactic-co-glycolic acid)). In some embodiments, the Poly(lactic-co-glycolic acid) consists of an 85:15 Lactic Acid:Glycolic acid ratio in the polymer.

In some embodiments, the polar solvent is selected from: Dichloromethane (DCM), Acetone, Acetonitrile, Chloroform, Dichloromethane (DCM), Dimethyl Sulfoxide (DMSO), Dimethyl Carbonate (DMC), Dimethylacetamide (DMAc), Dimethylformamide (DMF), Ethyl Acetate, Methanol, N-Methyl-2-Pyrrolidone (NMP), and Tetrahydrofuran (THF).

In some embodiments, the at least one active agent comprises one or more active agents selected from: growth factors, chemokines, cytokines, CD antigens, neurotrophins, and microRNAs.

In some embodiments, the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMP5), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGF5), Fibroblast growth factor 6 (FGF6), Fibroblast growth factor 7(FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDFS), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth Factor, Krüppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1), Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOXS, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-β), Transforming Growth Factor-β2 (TGF-β2), Transforming Growth Factor-β3 (TGF-β3), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor -D (VEGF-D).

In some embodiments, the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCLS), Eotaxin (CCL11,24,26), Exodus-2 (CCL21), Fractalkine (CX3CL1), GRO (CXCL1,2,3), HCC-1 (CCL14), I-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCL5), SDF (CXCL12), TARC (CCL17), CCL14, CCL19, CCL20, CCL27, CXCL13, and Thymus Expressed Chemokine (CCL25).

In some embodiments, the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36γ, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, and Wingless-Type MMTV Integration Site Family.

In some embodiments, the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CD5, CD8B, CD5L, CD68, CD55, CD7, CD73, CD58, CD74, CD80, CD79, CD84, CD93, CD99, CD164, and CD40L.

In some embodiments, the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, and Pleiotrophin.

In some embodiments, the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

In some embodiments, the buffer is selected from: Acetate buffers, ACES, ADA, AMP, AMPSO, AMPD, BES, Bicine, Bis-Tris, Bis-Tris Propane, CABS, CAPSO, CAPS, CHES, Citrate buffers, DIPSO, EPPS, Gly-Gly, HEPBS, HEPES, HEPPSO, PBS, PIPES, POPSO, MES, MOBS, MOPSO, MOPS, Sodium carbonate buffers, Sodium bicarbonate buffers, TABS, TAPS, TAPSO, TBS, TEA, TES, Tricine, and Tris.

In some embodiments, the carrier protein is selected from bovine serum albumin, human serum albumin, equine serum albumin, goat serum albumin, porcine serum albumin, rat serum albumin, mouse serum albumin, chicken serum albumin, and chicken white albumin.

In some embodiments, the salt of the bulk aqueous solution is selected from: chloride salts (e.g., NaCl), fluoride salts, phosphate salts, iron salts, carbonate salts, bicarbonate salts, sulfate salts, bisulfate salts, and potassium dichromate.

In some embodiments, the aqueous surfactant of the bulk aqueous solution is selected from: poly vinyl alcohol (PVA), polysorbate (e.g. Tweens), sorbitan esters (SPAN), poly(vinyl pyrrolidone) (PVP; also known as povidone, and poloxamers).

In some embodiments, the alcohol of the bulk aqueous solution is selected from: ethanol, propanol, n-butanol, 1-pentanol, and structural and functional isomers thereof.

In some embodiments, the method further comprises lyophilizing the isolated microspheres. In some embodiments, the method further comprises sieving the isolated microspheres and collecting the microspheres that are between about 10 μm to about 20 μm in diameter.

In some embodiments, the ratio of the polar solvent to the aqueous surfactant of the secondary emulsion is about 1:20 to about 1:1.

### BRIEF DESCRIPTION OF THE DRAWINGS

FIG. 1 shows a graphical summary of co-culture in vitro and rodent collagenase-induced in vivo models.

FIG. 2A-2E show SEM images of PLGA microspheres with PBS (FIG. 2A), microspheres loaded with FGF-18 (FIG. 2B), microspheres loaded with IGF-1 (FIG. 2C), microspheres loaded with TGF-β1 (FIG. 2D) and microspheres loaded with HGH (FIG. 2E). Larger images were captured using 1000× magnification (scale bar=10 μm) while smaller images in bottom right of each panel were captured under 10,000× magnification (scale bar=1 μm).

FIG. 3A-3C show cumulative release profiles of IGF-1 (FIG. 3A), HGH (FIG. 3B) and TGF-β1 (FIG. 3C). PLGA 85:15 Microspheres. n=4 batches measured in triplicate.

FIG. 4A-4D show that SASC exhibits anti-inflammatory and chondroprotective effects similar to ADSC. SASC reduced nitric oxide (NO) by Griess Reagent Assay (FIG. 4A), increased early chondrogenic marker SOX9 (FIG. 4B) and reduced catabolic gene expression of ADSMTSS (FIG. 4C). SASC also reduced the autocrine inflammatory response of PRG4 (FIG. 4D). ADSCs were able to attenuate ADAMTSS inflammatory response but did not have any effect on SOX9 or PRG4 or NO in the 3 day treatment window. *p<0.05; **p<0.01; ***p<0.001; ****p<0.0001. Complete statistical summary provided in Tables 2-5.

FIG. 5 shows progression of ipsilateral joint inflammation over the 9 week treatment period. Asterisk indicates initial point when ADSC and SASC exhibit lower swelling compared to blank and OA groups.

FIG. 6. Frontal sections of the Healthy Control (FIG. 6A), OA Control (FIG. 6B), ADSC group (FIG. 6C), blank group (FIG. 6D) and SASC group (FIG. 6E). Quantification of degenerated areas (FIG. 6F) shows SASC and ADSC attenuate progression of degenerative osteoarthritis. Arrows point to areas of significant cartilage thinning/degeneration. *p<0.05; **p<0.01. n=5. Complete statistical summary provided in Table 6.

FIG. 7. Surface heat map depicting tibial Young's modulus (MPa) of healthy (FIG. 7A), OA (FIG. 7B), ADSC (FIG. 7C), Blank (FIG. 7D) and SASC (FIG. FIG. 7E) treated joints (scale in FIG. 7F) and comparison of tibial young's modulus across groups (FIG. 7G). *p<0.05; **p<0.01; ***p<0.0001. Complete statistical summary provided in Table 7.

FIG. 8. Surface heat map depicting femoral Young's modulus (MPa) of healthy (FIG. 8A), OA (FIG. 8B), ADSC (FIG. 8C), Blank (FIG. 8D) and SASC (FIG. 8E) treated joints (scale in FIG. 8F) and comparison of femoral young's modulus across groups (FIG. 8G). *p<0.05; **p<0.01; ***p<0.0001. Complete statistical summary provided in Table 8.

FIG. 9 shows flow cytometry showing stemness of isolated ADSCs.

### DETAILED DESCRIPTION

The disclosure relates to synthetic artificial stem cells (SASCs), which as used herein, refer to a microsphere based, synthetic secretome delivery system that delivers a combination of active agents (e.g., growth factors found in the secretome) to a target site in a subject (e.g., an injury site). Synthetic artificial stem cells can be used as a replacement to stem cell therapy. For example, SASCs can replace adipose derived stem cells as one of the current therapies for osteoarthritis. Synthetic artificial stem cells (or SASCs) can be injected to release growth factors to have a comparable effect to that of stem cells. With the flexibility to manipulate the composition of growth factors injected into a subject, SASCs can be used as a therapeutic in any degenerative application. Such examples include, but are not limited to, Alzheimer's disease, spinal cord regeneration, muscular dystrophy, osteoporosis, osteoarthritis etc.

Cell therapy currently comes with the risk of adverse immune response. SASCs mitigate this risk along with providing a more potent delivery of the paracrine factors that make up the essence of the stem cell. The fabrication of SASCs also decreases the time for patient therapy by circumventing the need to expand and culture autologous cells in vitro before injecting them back into the patient. With SASC's increased shelf life, immediate therapy is possible. Another important benefit of SASCs is the ease of manufacturing. Taking away the culture requirements of traditional cells for individuals provides an opportunity to scale manufacturing to a point that has not been practical to do with cell therapy, improving the efficiency with which SASC therapy can be provided.

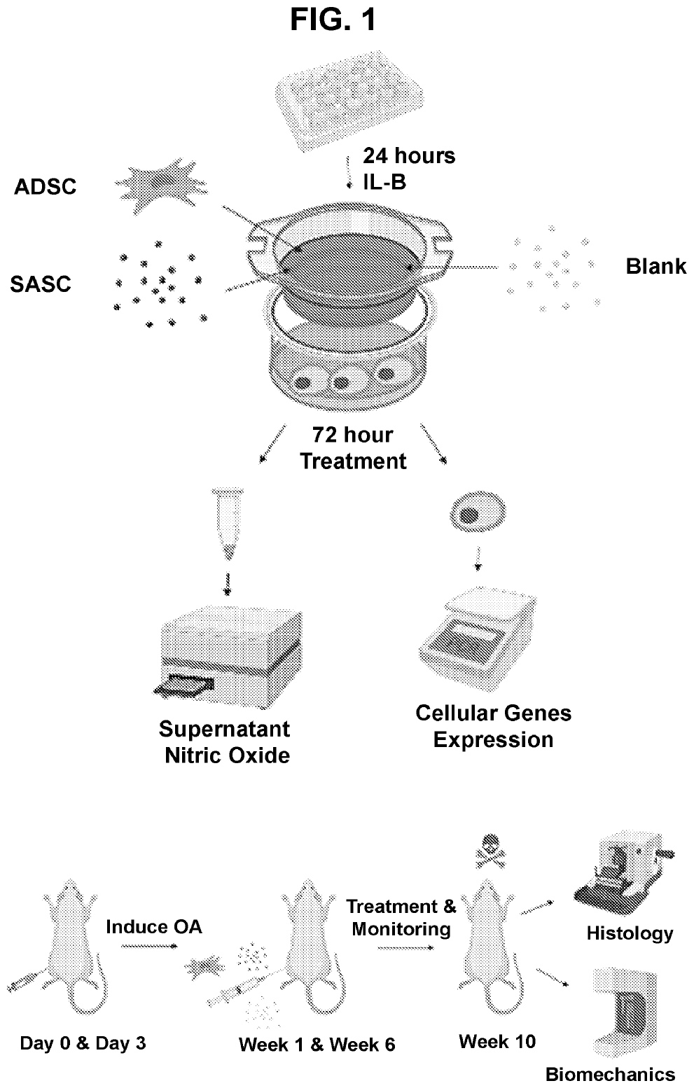

This disclosure describes the development of a SASC system exemplified in a proof of principle to attenuate osteoarthritis (OA) progression by tailoring factors abundant in the stromal stem cell secretome that have specifically an anabolic, chondroprotective and/or anti-inflammatory effect in the joint. As a pilot SASC composition, insulin-like growth factor 1 (IGF-1), transforming growth factor β1 (TGF-β1), fibroblast growth factor 18 (FGF-18) and human growth hormone (HGH) were combined and delivered using a PLGA 85:15 matrix. The anti-inflammatory and chondroprotective effects of SASC have been evaluated in comparison with the current treatment standard, ADSCs⁵, in both in vitro and in vivo osteoarthritis models (FIG. 1).

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R.I. Freshney. 1987. Liss, Inc. New York, NY), Gene Transfer and Expression Protocols, pp. 109-128, ed. E.J. Murray, The Humana Press Inc., Clifton, N.J.), and the Ambion 1998 Catalog (Ambion, Austin, TX).

All embodiments of any aspect of the disclosure can be used in combination, unless the context clearly dictates otherwise.

As used herein and unless otherwise indicated, the terms “a” and “an” are taken to mean “one”, “at least one” or “one or more”. Unless otherwise required by context, singular terms used herein shall include pluralities and plural terms shall include the singular. All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Additionally, the words “herein,” “above,” and “below” and words of similar import, when used in this application, shall refer to this application as a whole and not to any particular portions of the application.

As used herein, the term “about” encompasses insubstantial variations, such as values within a standard margin of error of measurement (e.g., SEM) of a stated value. For example, the term “about” as used herein when referring to a measurable value such as a parameter, an amount, a temporal duration, can encompass variations of +/−10% or less, +/−5% or less, or +/−1% or less or less of and from the specified value. Designation of a range of values includes all integers within or defining the range, and all subranges defined by integers within the range. As used herein, statistical significance means p≤0.05.

In one aspect, the disclosure provides a composition comprising one or more populations of microspheres, wherein each of the one or more populations of microspheres comprises at least one active agent. In some embodiments, the microspheres comprise a polymeric waxy or other protective material such as a natural, a semi-synthetic, or a synthetic polymer. The microspheres disclosed here are biodegradable polymeric microspheres that are used to encapsulate active agents. These microspheres mimic or have the potential to result in a greater therapeutic paracrine effect compared to stem cells, without the added risk of immunological response or the extra pro-inflammatory, antagonistic or inert factors/cytokines. The composition of active agents may be personalized to any disease and/or individual, therefore, adding a much needed element to therapeutics. The microspheres as disclosed here also allow for a controlled release of the active agents, which provides a preservative effect. In certain embodiments, the microspheres can comprise a biocompatible polymer selected from, but not limited to Poly(lactic-co-glycolic acid) (PLGA), Poly(lactic acid) (PLA), Poly(ϵ-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof In an embodiment, the biocompatible polymer comprises PLGA (Poly(lactic-co-glycolic acid)). Microspheres as defined herein refer to spherical microscopic particles that range in diameter from about 1 μm to about 1,000 μm. In some embodiments, the microspheres have a size diameter ranging from about 1 μm to about 100 μm. In an embodiment, the microspheres have a diameter size ranging from about 10 μm to about 20 μm. In some embodiments, the microspheres have a controlled release, which can be separated into three phases: (1) an initial burst with about 30% surface loaded protein released within about 1 day, (2) a diffusion controlled phase over the next about 10 days, and (3) an equilibrium controlled release until about 90% was released by about the 28^{th} day. In certain embodiments, the microspheres comprising the one or more active agents can be designed with controlled release patterns and loading efficiencies, formulated using different weight ratios and tailored to be a unique synthetic cell therapy for other degenerative diseases or regenerative applications.

As used herein, an “active agent” refers one or more biological factors and/or molecules that are secreted into the extracellular matrix and can play a role in a wide range of biological processes, including homeostasis, immunomodulation, inflammation, angiogenesis and ECM organization. The functions of the one or more active agents can be broken into four main functions: angiogenesis, anti-apoptosis, anti-fibrosis and anti-inflammation. Various secreted proteins make up the one or more active agents, and can include growth factors, angiogenic factors, cytokines, chemokines, and extracellular vesicles for transport of lipids and proteins. The composition of the active agents can be highly dynamic and can be based on the cell type and microenvironment in which it will be used, thus allowing for greater design flexibility and versatility. In some embodiments, the at least one active agent comprises one or more active agents selected from: growth factors, chemokines, cytokines, CD antigens, neurotrophins, and microRNAs (miRNAs).

As used herein, the term “growth factor” refers to a secreted biologically active molecule that can affect the growth of cells by stimulating cell proliferation, wound healing, and/or cellular differentiation. For example, a growth factor can refer to a secreted molecule that promotes or inhibits mitosis, or affects cellular differentiation. Growth factors can act on specific cell surface receptors that subsequently transmit their growth signals to other intracellular components and eventually result in altered gene expression. In some embodiments, the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMP5), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGFS), Fibroblast growth factor 6 (FGF6), Fibroblast growth factor 7(FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDFS), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth Factor, Kruppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1),Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOX5, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-β), Transforming Growth Factor-β2 (TGF-β2), Transforming Growth Factor-β3 (TGF-β3), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor -D (VEGF-D).

In some embodiments, the microsphere composition comprises about 1 ng to about 100 mg of one or more growth factors. For example, about 100 mg of one or more growth factors, about 90 mg of one or more growth factors, about 80 mg of one or more growth factors, about 70 mg of one or more growth factors, about 60 mg of one or more growth factors, about 50 mg of one or more growth factors, about 40 mg of one or more growth factors, about 30 mg of one or more growth factors, about 20 mg of one or more growth factors, about 10 mg of one or more growth factors, about 9 mg of one or more growth factors, about 8 mg of one or more growth factors, about 7 mg of one or more growth factors, about 6 mg of one or more growth factors, about 5 mg of one or more growth factors, about 4 mg of one or more growth factors, about 3 mg of one or more growth factors, about 2 mg of one or more growth factors, about 1 mg of one or more growth factors, about 0.9 mg of one or more growth factors, about 0.8 mg of one or more growth factors, about 0.7 mg of one or more growth factors, about 0.6 mg of one or more growth factors, about 0.5 mg of one or more growth factors, about 0.4 mg of one or more growth factors, about 0.3 mg of one or more growth factors, about 0.2 mg of one or more growth factors, about 0.1 mg of one or more growth factors, about 0.09 mg of one or more growth factors, about 0.08 mg of one or more growth factors, about 0.07 mg of one or more growth factors, about 0.06 mg of one or more growth factors, about 0.05 mg of one or more growth factors, about 0.04 mg of one or more growth factors, about 0.03 mg of one or more growth factors, about 0.02 mg of one or more growth factors, or about 0.01 mg of one or more growth factors. For example, about 1000 ng of one or more growth factors, about 900 ng of one or more growth factors, about 800 ng of one or more growth factors, about 700 ng of one or more growth factors, about 600 ng of one or more growth factors, about 500 ng of one or more growth factors, about 400 ng of one or more growth factors, about 300 ng of one or more growth factors, about 200 ng of one or more growth factors. In some embodiments, the microsphere composition comprises about 1 ng to about 100 ng of one or more growth factors. For example, about 100 ng of one or more growth factors, about 90 ng of one or more growth factors, about 80 ng of one or more growth factors, about 70 ng of one or more growth factors, about 60 ng of one or more growth factors, about 50 ng of one or more growth factors, about 40 ng of one or more growth factors, about 30 ng of one or more growth factors, about 20 ng of one or more growth factors, about 10 ng of one or more growth factors, about 9 ng of one or more growth factors, about 8 ng of one or more growth factors, about 7 ng of one or more growth factors, about 6 ng of one or more growth factors, about 5 ng of one or more growth factors, about 4 ng of one or more growth factors, about 3 ng of one or more growth factors, about 2 ng of one or more growth factors, or about 1 ng of one or more growth factors.

As used herein, the term “chemokine” refers to a large family of small (˜5-10 kDa), secreted proteins that signal through cell surface G protein-coupled receptors. For example, chemokines can stimulate the migration of cells (e.g., white blood cells). Chemokines can play a role in the development and homeostasis of the immune system, and are involved in protective or destructive immune and inflammatory responses. Chemokines can also stimulate a variety of other types of directed and undirected migratory behavior, such as haptotaxis, chemokinesis, and haptokinesis, in addition to inducing cell arrest or adhesion. In some embodiments, the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCLS), Eotaxin (CCL11,24,26), Exodus-2 (CCL21), Fractalkine (CX3CL1), GRO (CXCL1,2,3), HCC-1 (CCL14), 1-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCL5), SDF (CXCL12), TARC (CCL17), CCL14, CCL19, CCL20, CCL27, CXCL13, or and Thymus Expressed Chemokine (CCL25).

In some embodiments, the microsphere composition comprises about 1 ng to about 100 mg of one or more chemokines. For example, about 100 mg of one or more chemokines, about 90 mg of one or more chemokines, about 80 mg of one or more of chemokines, about 70 mg of one or more chemokines, about 60 mg of one or more chemokines, about 50 mg of one or more chemokines, about 40 mg of one or more chemokines, about 30 mg of one or more chemokines, about 20 mg of one or more chemokines, about 10 mg of one or more chemokines, about 9 mg of one or more chemokines, about 8 mg of one or more chemokines, about 7 mg of one or more chemokines, about 6 mg of one or more chemokines, about 5 mg of one or more chemokines, about 4 mg of one or more chemokines, about 3 mg of one or more chemokines, about 2 mg of one or more chemokines, about 1 mg of one or more chemokines, about 0.9 mg of one or more chemokines, about 0.8 mg of one or more chemokines, about 0.7 mg of one or more chemokines, about 0.6 mg of one or more chemokines actors, about 0.5 mg of one or more chemokines, about 0.4 mg of one or more chemokines, about 0.3 mg of one or more chemokines, about 0.2 mg of one or more chemokines, about 0.1 mg of one or more chemokines, about 0.09 mg of one or more chemokines, about 0.08 mg of one or more chemokines, about 0.07 mg of one or more chemokines, about 0.06 mg of one or more chemokines, about 0.05 mg of one or more chemokines, about 0.04 mg of one or more chemokines, about 0.03 mg of one or more chemokines, about 0.02 mg of one or more chemokines, or about 0.01 mg of one or more chemokines. For example, about 1000 ng of one or more chemokines, about 900 ng of one or more chemokines, about 800 ng of one or more chemokines, about 700 ng of one or more chemokines, about 600 ng of one or more chemokines, about 500 ng of one or more chemokines, about 400 ng of one or more chemokines, about 300 ng of one or more chemokines, about 200 ng of one or more chemokines. In some embodiments, the microsphere composition comprises about 1 ng to about 100 ng of one or more chemokines. For example, about 100 ng of one or more of chemokines, about 90 ng of one or more chemokines, about 80 ng of one or more chemokines, about 70 ng of one or more chemokines, about 60 ng of one or more chemokines, about 50 ng of one or more chemokines, about 40 ng of one or more chemokines, about 30 ng of one or more chemokines, about 20 ng of one or more chemokines, about 10 ng of one or more chemokines, about 9 ng of one or more chemokines, about 8 ng of one or more chemokines, about 7 ng of one or more chemokines, about 6 ng of one or more chemokines, about 5 ng of one or more chemokines, about 4 ng of one or more chemokines, about 3 ng of one or more chemokines, about 2 ng of one or more chemokines, or about 1 ng of one or more chemokines.

As used herein, the term “cytokine” refers to small (˜5-25 kDa) secreted proteins released by cells have a specific effect on the interactions and communications between cells. Cytokines can also include lymphokines (cytokines made by lymphocytes), monokines (cytokines made by monocytes), and interleukins (cytokines made by one leukocyte and acting on other leukocytes). Cytokines can act on the cells that secrete them (autocrine action), on nearby cells (paracrine action), or in some instances on distant cells (endocrine action). Cytokines can affect disease pathogenesis, non-specific response to infection, specific response to antigen, changes in cognitive functions, and progression of the degenerative processes of aging. In addition, cytokines can be part of stem cell differentiation, vaccine efficacy and allograft rejection. In some embodiments, the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36γ, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, or and Wingless-Type MMTV Integration Site Family.

In some embodiments, the microsphere composition comprises about 1 ng to about 100 mg of one or more cytokines. For example, about 100 mg of one or more cytokines, about 90 mg of one or more cytokines, about 80 mg of one or more cytokines, about 70 mg of one or more cytokines, about 60 mg of one or more cytokines, about 50 mg of one or more cytokines, about 40 mg of one or more cytokines, about 30 mg of one or more cytokines, about 20 mg of one or more cytokines, about 10 mg of one or more cytokines, about 9 mg of one or more cytokines, about 8 mg of one or more cytokines, about 7 mg of one or more cytokines, about 6 mg of one or more cytokines, about 5 mg of one or more cytokines, about 4 mg of one or more cytokines, about 3 mg of one or more cytokines, about 2 mg of one or more cytokines, about 1 mg of one or more cytokines, about 0.9 mg of one or more cytokines, about 0.8 mg of one or more cytokines, about 0.7 mg of one or more cytokines, about 0.6 mg of one or more cytokines, about 0.5 mg of one or more cytokines, about 0.4 mg of one or more cytokines, about 0.3 mg of one or more cytokines, about 0.2 mg of one or more cytokines, about 0.1 mg of one or more cytokines, about 0.09 mg of one or more cytokines, about 0.08 mg of one or more cytokines, about 0.07 mg of one or more cytokines, about 0.06 mg of one or more cytokines, about 0.05 mg of one or more cytokines, about 0.04 mg of one or more cytokines, about 0.03 mg of one or more cytokines, about 0.02 mg of one or more cytokines, or about 0.01 mg of one or more cytokines. For example, about 1000 ng of one or more cytokines, about 900 ng of one or more cytokines, about 800 ng of one or more cytokines, about 700 ng of one or more cytokines, about 600 ng of one or more cytokines, about 500 ng of one or more cytokines, about 400 ng of one or more cytokines, about 300 ng of one or more cytokines, about 200 ng of one or more cytokines. In some embodiments, the microsphere composition comprises about 1 ng to about 100 ng of one or more cytokines. For example, about 100 ng of one or more cytokines, about 90 ng of one or more cytokines, about 80 ng of one or more cytokines, about 70 ng of one or more cytokines, about 60 ng of one or more cytokines, about 50 ng of one or more cytokines, about 40 ng of one or more cytokines, about 30 ng of one or more cytokines, about 20 ng of one or more cytokines, about 10 ng of one or more cytokines, about 9 ng of one or more cytokines, about 8 ng of one or more cytokines, about 7 ng of one or more cytokines, about 6 ng of one or more cytokines, about 5 ng of one or more cytokines, about 4 ng of one or more cytokines, about 3 ng of one or more cytokines, about 2 ng of one or more cytokines, or about 1 ng of one or more cytokines.

As used herein, the term “CD antigen” refers to CD (cluster of differentiation) antigens that are cell surface molecules expressed on leukocytes and other cells relevant for the immune system. CD antigens are also known as cluster of designation or classification determinant and, CD molecules can act in numerous ways, often acting as receptors or ligands important to the cell. For example, a signal cascade can be initiated, altering the behavior of the cell. Some CD antigens do not play a role in cell signaling, but have other functions, for example such as cell adhesion. In some embodiments, the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CDS, CD8B, CDSL, CD68, CD55, CD7, CD73, CD58, CD74, CD80, CD79, CD84, CD93, CD99, CD164, or and CD40L.

In some embodiments, the microsphere composition comprises about 1 ng to about 100 mg of one or more CD antigens. For example, about 100 mg of one or more CD antigens, about 90 mg of one or more CD antigens, about 80 mg of one or more CD antigens, about 70 mg of one or more CD antigens, about 60 mg of one or more CD antigens, about 50 mg of one or more CD antigens, about 40 mg of one or more CD antigens, about 30 mg of one or more CD antigens, about 20 mg of one or more CD antigens, about 10 mg of one or more CD antigens, about 9 mg of one or more CD antigens, about 8 mg of one or more CD antigens, about 7 mg of one or more CD antigens, about 6 mg of one or more CD antigens, about 5 mg of one or more CD antigens, about 4 mg of one or more CD antigens, about 3 mg of one or more CD antigens, about 2 mg of one or more CD antigens, about 1 mg of one or more CD antigens, about 0.9 mg of one or more CD antigens, about 0.8 mg of one or more CD antigens, about 0.7 mg of one or more CD antigens, about 0.6 mg of one or more CD antigens, about 0.5 mg of one or more CD antigens, about 0.4 mg of one or more CD antigens, about 0.3 mg of one or more CD antigens, about 0.2 mg of one or more CD antigens, about 0.1 mg of one or more CD antigens, about 0.09 mg of one or more CD antigens, about 0.08 mg of one or more CD antigens, about 0.07 mg of one or more CD antigens, about 0.06 mg of one or more CD antigens, about 0.05 mg of one or more CD antigens, about 0.04 mg of one or more CD antigens, about 0.03 mg of one or more CD antigens, about 0.02 mg of one or more CD antigens, or about 0.01 mg of one or more CD antigens. For example, about 1000 ng of one or more CD antigens, about 900 ng of one or more CD antigens, about 800 ng of one or more CD antigens, about 700 ng of one or more CD antigens, about 600 ng of one or more CD antigens, about 500 ng of one or more CD antigens, about 400 ng of one or more CD antigens, about 300 ng of one or more CD antigens, about 200 ng of one or more CD antigens. In some embodiments, the microsphere composition comprises about 1 ng to about 100 ng of one or more CD antigens. For example, about 100 ng of one or more CD antigens, about 90 ng of one or more CD antigens, about 80 ng of one or more CD antigens, about 70 ng of one or more CD antigens, about 60 ng of one or more CD antigens, about 50 ng of one or more CD antigens, about 40 ng of one or more CD antigens, about 30 ng of one or more CD antigens, about 20 ng of one or more CD antigens, about 10 ng of one or more CD antigens, about 9 ng of one or more CD antigens, about 8 ng of one or more CD antigens, about 7 ng of one or more CD antigens, about 6 ng of one or more CD antigens, about 5 ng of one or more CD antigens, about 4 ng of one or more CD antigens, about 3 ng of one or more CD antigens, about 2 ng of one or more CD antigens, or about 1 ng of one or more CD antigens.

As used herein, the term “neurotrophin” refers to a family of secreted proteins that are important regulators of neural survival, development, function, and plasticity. In some embodiments, the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, or and Pleiotrophin.

In some embodiments, the microsphere composition comprises about 1 ng to about 100 mg of one or more neurotrophins. For example, about 100 mg of one or more neurotrophins, about 90 mg of one or more neurotrophins, about 80 mg of one or more neurotrophins, about 70 mg of one or more neurotrophins, about 60 mg of one or more neurotrophins, about 50 mg of one or more neurotrophins, about 40 mg of one or more neurotrophins, about 30 mg of one or more neurotrophins, about 20 mg of one or more neurotrophins, about 10 mg of one or more neurotrophins, about 9 mg of one or more neurotrophins, about 8 mg of one or more neurotrophins, about 7 mg of one or more neurotrophins, about 6 mg of one or more neurotrophins, about 5 mg of one or more neurotrophins, about 4 mg of one or more neurotrophins, about 3 mg of one or more neurotrophins, about 2 mg of one or more neurotrophins, about 1 mg of one or more neurotrophins, about 0.9 mg of one or more neurotrophins, about 0.8 mg of one or more neurotrophins, about 0.7 mg of one or more neurotrophins, about 0.6 mg of one or more neurotrophins, about 0.5 mg of one or more neurotrophins, about 0.4 mg of one or more neurotrophins, about 0.3 mg of one or more neurotrophins, about 0.2 mg of one or more neurotrophins, about 0.1 mg of one or more neurotrophins, about 0.09 mg of one or more neurotrophins, about 0.08 mg of one or more neurotrophins, about 0.07 mg of one or more neurotrophins, about 0.06 mg of one or more neurotrophins, about 0.05 mg of one or more neurotrophins, about 0.04 mg of one or more neurotrophins, about 0.03 mg of one or more neurotrophins, about 0.02 mg of one or more neurotrophins, or about 0.01 mg of one or more neurotrophins. For example, about 1000 ng of one or more neurotrophins, about 900 ng of one or more neurotrophins, about 800 ng of one or more neurotrophins, about 700 ng of one or more neurotrophins, about 600 ng of one or more neurotrophins, about 500 ng of one or more neurotrophins, about 400 ng of one or more neurotrophins, about 300 ng of one or more neurotrophins, about 200 ng of one or more neurotrophins. In some embodiments, the microsphere composition comprises about 1 ng to about 100 ng of one or more neurotrophins. For example, about 100 ng of one or more neurotrophins, about 90 ng of one or more neurotrophins, about 80 ng of one or more neurotrophins, about 70 ng of one or more neurotrophins, about 60 ng of one or more neurotrophins, about 50 ng of one or more neurotrophins, about 40 ng of one or more neurotrophins, about 30 ng of one or more neurotrophins, about 20 ng of one or more neurotrophins, about 10 ng of one or more neurotrophins, about 9 ng of one or more neurotrophins, about 8 ng of one or more neurotrophins, about 7 ng of one or more neurotrophins, about 6 ng of one or more neurotrophins, about 5 ng of one or more neurotrophins, about 4 ng of one or more neurotrophins, about 3 ng of one or more neurotrophins, about 2 ng of one or more neurotrophins, or about 1 ng of one or more neurotrophins.

As used herein, the terms “microRNA” and “miRNA” refer to a family of small non-protein-coding RNAs with a single strand of about 18-25 nucleotides that regulate multiple target genes at the post-transcriptional level, MiRNAs can function in RNA silencing and post-transcriptional regulation of gene expression. In some embodiments, the miRNA can be involved in cartilage production, neural processes, bone development, and/or skeletal muscle healing. In some embodiments, the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

In some embodiments, the microsphere composition comprises about 0.1 mg to about 300 mg of one or more miRNAs. For example, about 300 mg of one or more miRNAs, about 275 mg of one or more miRNAs, about 250 mg of one or more miRNAs, about 225 mg of one or more miRNAs, about 200 mg of one or more miRNAs, about 175 mg of one or more miRNAs, about 150 mg of one or more miRNAs, about 125 mg of one or more miRNAs, about 100 mg of one or more miRNAs, about 90 mg of one or more miRNAs, about 80 mg of one or more miRNAs, about 75 mg of one or more miRNAs, about 70 mg of one or more miRNAs, about 60 mg of one or more miRNAs, about 50 mg of one or more miRNAs, about 40 mg of one or more miRNAs, about 30 mg of one or more miRNAs, about 20 mg of one or more miRNAs, about 10 mg of one or more miRNAs, about 9 mg of one or more miRNAs, about 8 mg of one or more miRNAs, about 7 mg of one or more miRNAs, about 6 mg of one or more miRNAs, about 5 mg of one or more miRNAs, about 4 mg of one or more miRNAs, about 3 mg of one or more miRNAs, about 2 mg of one or more miRNAs, about 1 mg of one or more miRNAs, about 0.9 mg of one or more miRNAs, about 0.8 mg of one or more miRNAs, about 0.7 mg of one or more miRNAs, about 0.6 mg of one or more miRNAs, about 0.5 mg of one or more miRNAs, about 0.4 mg of one or more miRNAs, about 0.3 mg of one or more miRNAs, about 0.2 mg of one or more miRNAs, or about 0.1 mg of one or more miRNAs. For example, about 1000 ng of one or more miRNAs, about 900 ng of one or more miRNAs, about 800 ng of one or more miRNAs, about 700 ng of one or more miRNAs, about 600 ng of one or more miRNAs, about 500 ng of one or more miRNAs, about 400 ng of one or more miRNAs, about 300 ng of one or more miRNAs, about 200 ng of one or more miRNAs.

In some embodiments, the microsphere composition comprises about 0.01 mg/kg to about 5 mg/kg of one or more of microRNAs. For example, about 5 mg/kg of one or more of microRNAs, about 4 mg/kg of one or more microRNAs, about 3 mg/kg of one or more of microRNAs, about 2 mg/kg of one or more microRNAs, about 1 mg/kg of one or more microRNAs, about 0.9 mg/kg of one or more microRNAs, about 0.8 mg/kg of one or more microRNAs, about 0.7 mg/kg of one or more microRNAs, about 0.6 mg/kg of one or more microRNAs, about 0.5 mg/kg of one or more microRNAs, about 0.4 mg/kg of one or more microRNAs, about 0.3 mg/kg of one or more microRNAs, about 0.2 mg/kg of one or more microRNAs, about 0.1 mg/kg of one or more microRNAs, about 0.09 mg/kg of one or more microRNAs, about 0.08 mg/kg of one or more microRNAs, about 0.07 mg/kg of one or more microRNAs, about 0.06 mg/kg of one or more microRNAs, about 0.05 mg/kg of one or more microRNAs, about 0.04 mg/kg of one or more microRNAs, about 0.03 mg/kg of one or more microRNAs, about 0.02 mg/kg of one or more microRNAs, or about 0.01 mg/kg of one or more microRNAs.

In certain embodiments, the composition can comprise two or more populations of microspheres and each population of microspheres comprises a single active agent. In a non-limiting example, a composition can comprise three unique populations of microspheres, wherein the first population of microspheres comprises, for example, IGF-1, the second population of microspheres comprises, for example, TGF-β, and the third population of microspheres comprises, for example, HGH. In another non-limiting example, a composition can comprise four unique populations of microspheres, wherein the first population of microspheres comprises, for example, IGF-1, the second population of microspheres comprises, for example, TGF-β, the third population of microspheres comprises, for example, HGH, and the fourth population of microspheres comprises, for example, FGF-18. In yet another non-limiting example, a composition can comprise five unique populations of microspheres, wherein the first population of microspheres comprises, for example, IGF-1, the second population of microspheres comprises, for example, TGF-β, the third population of microspheres comprises, for example, HGH, the fourth population of microspheres comprises, for example, FGF-18, and the fifth population of microspheres comprises, for example, VEGF.

In certain embodiments, the composition can comprise two or more active agents in a single population of microspheres. In a non-limiting example, a composition can comprise three active agents in a single population of microspheres, wherein the first active agent is, for example, IGF-1, the second active agent is, for example, TGF-β, and the third active agent is, for example, HGH. In another non-limiting example, a composition can comprise four active agents in a single population of microspheres, wherein the first active agent is, for example, IGF-1, the second active agent is, for example, TGF-β, the third active agent is, for example, HGH, and the fourth active agent is, for example, FGF-18. In yet another non-limiting example, a composition can comprise five active agents in a single population of microspheres, wherein the first active agent is, for example, IGF-1, the second active agent is, for example, TGF-β, the third active agent is, for example, HGH, the fourth active agent is, for example, FGF-18, and the fifth active agent is, for example, VEGF.

In certain embodiments, the composition can comprise two or more populations of microspheres, wherein one or more populations of the microspheres comprises a single active agent, while one more additional populations of microspheres can comprise two or more active agents in a single population of microspheres. In a non-limiting example, a composition can comprise two unique populations of microspheres, wherein the first population of microspheres comprises, for example, IGF-1, and the second population of microspheres comprises, for example, TGF-β, HGH, FGF-18, and VEGF. In another non-limiting example, a composition can comprise three unique populations of microspheres, wherein the first population of microspheres comprises, for example, IGF-1, the second population of microspheres comprises, for example, TGF-β, and the third population of microspheres comprises, for example, HGH and TGF-β. In another non-limiting example, a composition can comprise three unique populations of microspheres, wherein the first population of microspheres comprises, for example, IGF-1, the second population of microspheres comprises, for example, TGF-β and VEGF, and the third population of microspheres comprises, for example, HGH and TGF-β. In yet another non-limiting example, a composition can comprise four unique populations of microspheres, wherein the first population of microspheres comprises, for example, IGF-1, the second population of microspheres comprises, for example, TGF-β, the third population of microspheres comprises HGH, and the fourth population of microspheres comprises, for example, FGF-18 and VEGF.

In certain embodiments, the at least one active agent comprises VEGF, IGF-1, TGF-β, HGH, and FGF-18. In certain embodiments, the at least one active agent comprises IGF-1, TGF-β, HGH, and FGF-18. In certain embodiments, the at least one active agent comprises VEGF, TGF-β, and BMP2. In certain embodiments, the at least one active agent comprises Myostatin, IGF-1, and Growth differentiation factor 11. In certain embodiments, the at least one active agent comprises Oct-4, SOX2, KLF4, and c-Myc.

In some embodiments, the at least one active agent is mixed with a carrier protein. In some embodiments, the at least one active agent is mixed with a combination of two or more carrier proteins. A carrier protein refers to a protein that helps increase the loading efficiency of the one or more active agents in the microspheres. In some embodiments, a carrier protein can refer to a protein that aids the transport of the one or more active agents through intracellular fluid, through extracellular fluid, or across cell membranes. Non-limiting examples of carrier proteins are bovine serum albumin, human serum albumin, equine serum albumin, goat serum albumin, porcine serum albumin, rat serum albumin, mouse serum albumin, chicken serum albumin, and chicken white albumin. In certain embodiments, an active agent can be combined in a solution comprising about 0 mg/mL to about 40 mg/mL of a carrier protein. In certain embodiments, an active agent can be combined in a solution comprising about 1 mg/mL of a carrier protein. In certain embodiments, an active agent can be combined in a solution comprising about 2 mg/mL a carrier protein. In certain embodiments, an active agent can be combined in a solution comprising about 3 mg/mL of a carrier protein. In certain embodiments, an active agent can be combined in a solution comprising about 4 mg/mL of a carrier protein. In certain embodiments, an active agent can be combined in a solution comprising about 5 mg/mL of a carrier protein. In certain embodiments, an active agent can be combined in a solution comprising about 6 mg/mL of a carrier protein. In certain embodiments, an active agent can be combined in a solution comprising about 7 mg/mL of a carrier protein. In certain embodiments, an active agent can be combined in a solution comprising about 8 mg/mL of a carrier protein. In certain embodiments, an active agent can be combined in a solution comprising about 9 mg/mL of a carrier protein. In certain embodiments, an active agent can be combined in a solution comprising about 10 mg/mL of a carrier protein. In certain embodiments, an active agent can be combined in a solution comprising about 20 mg/mL of a carrier protein. In certain embodiments, an active agent can be combined in a solution comprising about 30 mg/mL of a carrier protein. In certain embodiments, an active agent can be combined in a solution comprising about 40 mg/mL of a carrier protein. In certain embodiments, an active agent can be combined in a solution comprising about 1% to about 10% of a carrier protein (w/v). In certain embodiments, an active agent can be combined in a solution comprising about 1% a carrier protein (w/v). In certain embodiments, an active agent can be combined in a solution comprising about 2% a carrier protein (w/v). In certain embodiments, an active agent can be combined in a solution comprising about 3% a carrier protein (w/v). In certain embodiments, an active agent can be combined in a solution comprising about 4% a carrier protein (w/v). In certain embodiments, an active agent can be combined in a solution comprising about 5% a carrier protein (w/v). In certain embodiments, an active agent can be combined in a solution comprising about 6% a carrier protein (w/v). In certain embodiments, an active agent can be combined in a solution comprising about 7% a carrier protein (w/v). In certain embodiments, an active agent can be combined in a solution comprising about 8% a carrier protein (w/v). In certain embodiments, an active agent can be combined in a solution comprising about 9% a carrier protein (w/v). In certain embodiments, an active agent can be combined in a solution comprising about 10% a carrier protein (w/v).

In certain embodiments, the composition further comprises one or more delivery vehicles, diluents, excipients, pharmaceutical adjuvants, stimulants, and/or stabilizers. For example, a composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides--preferably sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

In some embodiments, the composition is a “pharmaceutical composition” or “therapeutic composition.” The terms “pharmaceutical composition” or “therapeutic composition” as used herein refer to a compound or composition capable of inducing a desired therapeutic effect when properly administered to a subject. For example, in a patient with a degenerative condition such as osteoarthritis, administration of a therapeutic composition (for example, microspheres comprising IGF-1, TGF-β, HGH, and FGF-18) would result in stimulation of ECM synthesis as well as a reduction in inflammation to disrupt the cycle of inflammation/degeneration. In another example, in a subject with skeletal muscle degeneration, administration of a therapeutic composition (for example, microspheres comprising Myostatin, IGF-1, and Growth differentiation factor 11) would result in muscle regeneration.

In another aspect, this disclosure provides for methods for treating a subject, comprising administering an effective dose of the compositions as disclosed herein. In certain embodiments of the methods for treating a subject, an effective dose can be a pharmaceutical composition comprising the microsphere compositions as disclosed herein. In certain non-limiting examples, the subject is suspected of having or a degenerative disease or injury. A degenerative disease can be the result of a continuous process based on degenerative cell changes, affecting tissues or organs, which will increasingly deteriorate over time, and the compositions disclosed herein can be used to treat degenerative diseases by providing a disease modifying therapy leading to regeneration of native tissue. In certain non-limiting examples, the subject is suspected of having or has a degenerative nerve disease such as Alzheimer's disease, Amyotrophic lateral sclerosis, Friedreich's ataxia, Huntington's disease, Lewy body disease, Parkinson's disease, or Spinal muscular atrophy. In some embodiments, the subject is suspected of having or has Alzheimer's disease, spinal cord injury, macular degeneration, multiple sclerosis, muscular dystrophy, arthritis, osteoporosis, and/or osteoarthritis, and the method serves to treat Alzheimer's disease, spinal cord injury, macular degeneration, multiple sclerosis, muscular dystrophy, arthritis, osteoporosis, and/or osteoarthritis. The term “subject” as used herein includes human and non-human animal subjects. In some embodiments, a subject can be any suitable mammalian subject, including but not limited to a horse, rabbit, mouse, cow, dog, cat, sheep, goat, or human. In an embodiment, the mammalian subject may be a human. As used herein, the terms “treatment” or “treat” can refer to therapeutic treatment and/or prophylactic or preventative measures. The effect of the treatment may be therapeutic in terms of a partial or complete cure for a degenerative disease and/or adverse symptoms attributable to the degenerative disease. Those in need of treatment include those having a degenerative disorder as well as those prone to have a degenerative disorder or those in which a degenerative disorder is to be prevented. In certain embodiments, the compositions as disclosed herein are administered to a subject in need of treatment once per month, twice per month, three times per month, four times per month, or more. In certain embodiments, the compositions as disclosed herein are administered to a subject in need of treatment about every 5 weeks. The appropriate dose of the one or more active agents in the microspheres, as well as the frequency, and duration of treatment can be modified to address the particular needs of a particular subject by taking into account factors including, but not limited to, the age, gender, weight, and health of the subject; the severity, extent, and type of the degenerative condition. In some embodiments, the length of treatment can be less than 1 month to 12 months, or more than 12 months. In certain embodiments, treatment duration can be indefinite. In some embodiments, treatment duration can be until disease remission.

The term “effective amount” and when used in reference to the microsphere compositions comprising one or more active agents as disclosed herein, refer to an amount or dosage sufficient to produce a desired therapeutic result. More specifically, an effective amount is an amount of microsphere compositions comprising one or more active agents sufficient to prevent and/or inhibit, for some period of time, one or more of the clinically defined pathological processes associated with the degenerative disease being treated. The effective amount may vary depending on the dosage of the one or more active agents that are being used, and also depends on a variety of factors and conditions related to the subject being treated and the severity of the disease. For example, factors such as the age, weight, and health of the subject as well as dose response curves and toxicity data obtained in preclinical animal work would be among those factors considered in determining the effective amount to be administered. The determination of an effective amount of a given pharmaceutical composition is within the ability of those skilled in the art.

In some embodiments, the effective amount of the microsphere compositions comprising one or more active agents is administered as a single dose or in multiple doses. In some embodiments, the effective amount of the microsphere compositions comprising one or more active agents is administered as a controlled release formulation. In certain embodiments, the effective amount of the microsphere compositions comprising one or more active agents is administered one time per month, two times per month, three times per month, or four times per month. Dosing frequency will depend upon the pharmacokinetic parameters of the composition being used. Typically, a clinician will administer the composition until a dosage is reached that achieves the desired effect. In some embodiments, the composition is administered about every 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 days. In certain embodiments, the composition is administered about every 35, 28, 24, 12, 8, 6, or 4 days. In certain embodiments, the length of treatment can be less than 1 month to 12 months, or more than 12 months. For example, the length of treatment can be from about 1 months to about 4 months, from about 2 months to about 6 months, from about 4 months to about 8 months, from about 1 month to about 3 months, from about 2 months to about 4 months, from about 3 months to about 6 months, from about 6 months to about 12 months, or more than 12 months. In some embodiments, treatment duration can be until disease remission, for example, from about 1 month to about 24 months, or more than 24 months. In some embodiments, the subject is treated for about 1 month, about 2 months, about 3 months, about 4 months, about 5 months, about 6 months, about 7 months, about 8 months, about 9 months, about 10 months, about 11 months, about 1 year, about 2 years, or more than 2 years. In certain embodiments, the subject is treated indefinitely.

In some embodiments, the microsphere compositions comprising one or more active agents are administered by injection (subcutaneous injection, subdermal injection, intramuscular injection, depot administration, or intravenous injection), orally, parenterally, transdermally, topically, transmucosally, by inhalation, by suppository, by buccal delivery, by sublingual delivery, by ophthalmic delivery. In some embodiments, the microsphere compositions comprising one or more active agents can a controlled release composition (i.e., designed to slowly release over an extended period of time with a reduction of the peak/trough ratio compared to standard release). In some embodiments, a controlled release maintains release of the one or more active agents over a sustained period at a nearly constant rate. In some embodiments, controlled release compositions are dosage forms designed to release the one or more active agents at a predetermined rate in order to maintain a constant concentration for a specific period of time with minimum side effects.

In another aspect, this disclosure provides for a method for preparing a composition comprising microspheres and at least one active agent, the method comprising:

(a) dissolving a polymer in a polar solvent and mixing to prepare a dissolved polymer;

(b) mixing the at least one active agent with a buffer and a carrier protein to prepare a diluted active agent;

(c) combining the dissolved polymer with the diluted active agent and mixing to prepare a primary emulsion;

(d) mixing the primary emulsion with an aqueous surfactant to make a secondary emulsion;

(e) mixing the secondary emulsion in a bulk aqueous solution comprising a salt, an aqueous surfactant, and an alcohol until the polar solvent is evaporated; and

(f) isolating the microspheres.

In some embodiments of the method, the polymer is selected from: Poly(lactic-co-glycolic acid) (PLGA), Poly(lactic acid) (PLA), Poly(ϵ-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof.

In some embodiments of the method, the polar solvent is selected from: Dichloromethane (DCM), Acetone, Acetonitrile, Chloroform, Dichloromethane (DCM), Dimethyl Sulfoxide (DMSO), Dimethyl Carbonate (DMC), Dimethylacetamide (DMAc), Dimethylformamide (DMF), Ethyl Acetate, Methanol, N-Methyl-2-Pyrrolidone (NMP), and Tetrahydrofuran (THF).

In some embodiments of the method, the at least one active agent comprises one or more active agents selected from: growth factors, chemokines, cytokines, CD antigens, neurotrophins, and microRNAs.

In some embodiments of the method, the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMP5), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGFS), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDFS), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth

Factor, Kruppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1), Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOX5, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-(3), Transforming Growth Factor-β2 (TGF-(β2), Transforming Growth Factor-β3 (TGF-(β3), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor -D (VEGF-D).

In some embodiments, the microsphere composition comprises about 1 ng to about 100 mg of one or more growth factors. For example, about 100 mg of one or more growth factors, about 90 mg of one or more growth factors, about 80 mg of one or more growth factors, about 70 mg of one or more growth factors, about 60 mg of one or more growth factors, about 50 mg of one or more growth factors, about 40 mg of one or more growth factors, about 30 mg of one or more growth factors, about 20 mg of one or more growth factors, about 10 mg of one or more growth factors, about 9 mg of one or more growth factors, about 8 mg of one or more growth factors, about 7 mg of one or more growth factors, about 6 mg of one or more growth factors, about 5 mg of one or more growth factors, about 4 mg of one or more growth factors, about 3 mg of one or more growth factors, about 2 mg of one or more growth factors, about 1 mg of one or more growth factors, about 0.9 mg of one or more growth factors, about 0.8 mg of one or more growth factors, about 0.7 mg of one or more growth factors, about 0.6 mg of one or more growth factors, about 0.5 mg of one or more growth factors, about 0.4 mg of one or more growth factors, about 0.3 mg of one or more growth factors, about 0.2 mg of one or more growth factors, about 0.1 mg of one or more growth factors, about 0.09 mg of one or more growth factors, about 0.08 mg of one or more growth factors, about 0.07 mg of one or more growth factors, about 0.06 mg of one or more growth factors, about 0.05 mg of one or more growth factors, about 0.04 mg of one or more growth factors, about 0.03 mg of one or more growth factors, about 0.02 mg of one or more growth factors, or about 0.01 mg of one or more growth factors. For example, about 1000 ng of one or more growth factors, about 900 ng of one or more growth factors, about 800 ng of one or more growth factors, about 700 ng of one or more growth factors, about 600 ng of one or more growth factors, about 500 ng of one or more growth factors, about 400 ng of one or more growth factors, about 300 ng of one or more growth factors, about 200 ng of one or more growth factors. In some embodiments, the microsphere composition comprises about 1 ng to about 100 ng of one or more growth factors. For example, about 100 ng of one or more growth factors, about 90 ng of one or more growth factors, about 80 ng of one or more growth factors, about 70 ng of one or more growth factors, about 60 ng of one or more growth factors, about 50 ng of one or more growth factors, about 40 ng of one or more growth factors, about 30 ng of one or more growth factors, about 20 ng of one or more growth factors, about 10 ng of one or more growth factors, about 9 ng of one or more growth factors, about 8 ng of one or more growth factors, about 7 ng of one or more growth factors, about 6 ng of one or more growth factors, about 5 ng of one or more growth factors, about 4 ng of one or more growth factors, about 3 ng of one or more growth factors, about 2 ng of one or more growth factors, or about 1 ng of one or more growth factors.

In some embodiments of the method, the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCL5), Eotaxin (CCL11,24,26), Exodus-2 (CCL21), Fractalkine (CX3CL1), GRO (CXCL1,2,3), HCC-1 (CCL14), I-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCL5), SDF (CXCL12), TARC (CCL17), CCL14, CCL19, CCL20, CCL27, CXCL13, and Thymus Expressed Chemokine (CCL25).

In some embodiments, the microsphere composition comprises about 1 ng to about 100 mg of one or more chemokines. For example, about 100 mg of one or more chemokines, about 90 mg of one or more chemokines, about 80 mg of one or more of chemokines, about 70 mg of one or more chemokines, about 60 mg of one or more chemokines, about 50 mg of one or more chemokines, about 40 mg of one or more chemokines, about 30 mg of one or more chemokines, about 20 mg of one or more chemokines, about 10 mg of one or more chemokines, about 9 mg of one or more chemokines, about 8 mg of one or more chemokines, about 7 mg of one or more chemokines, about 6 mg of one or more chemokines, about 5 mg of one or more chemokines, about 4 mg of one or more chemokines, about 3 mg of one or more chemokines, about 2 mg of one or more chemokines, about 1 mg of one or more chemokines, about 0.9 mg of one or more chemokines, about 0.8 mg of one or more chemokines, about 0.7 mg of one or more chemokines, about 0.6 mg of one or more chemokines actors, about 0.5 mg of one or more chemokines, about 0.4 mg of one or more chemokines, about 0.3 mg of one or more chemokines, about 0.2 mg of one or more chemokines, about 0.1 mg of one or more chemokines, about 0.09 mg of one or more chemokines, about 0.08 mg of one or more chemokines, about 0.07 mg of one or more chemokines, about 0.06 mg of one or more chemokines, about 0.05 mg of one or more chemokines, about 0.04 mg of one or more chemokines, about 0.03 mg of one or more chemokines, about 0.02 mg of one or more chemokines, or about 0.01 mg of one or more chemokines. For example, about 1000 ng of one or more chemokines, about 900 ng of one or more chemokines, about 800 ng of one or more chemokines, about 700 ng of one or more chemokines, about 600 ng of one or more chemokines, about 500 ng of one or more chemokines, about 400 ng of one or more chemokines, about 300 ng of one or more chemokines, about 200 ng of one or more chemokines. In some embodiments, the microsphere composition comprises about 1 ng to about 100 ng of one or more chemokines.

For example, about 100 ng of one or more of chemokines, about 90 ng of one or more chemokines, about 80 ng of one or more chemokines, about 70 ng of one or more chemokines, about 60 ng of one or more chemokines, about 50 ng of one or more chemokines, about 40 ng of one or more chemokines, about 30 ng of one or more chemokines, about 20 ng of one or more chemokines, about 10 ng of one or more chemokines, about 9 ng of one or more chemokines, about 8 ng of one or more chemokines, about 7 ng of one or more chemokines, about 6 ng of one or more chemokines, about 5 ng of one or more chemokines, about 4 ng of one or more chemokines, about 3 ng of one or more chemokines, about 2 ng of one or more chemokines, or about 1 ng of one or more chemokines.

In some embodiments of the method, the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36y, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, and Wingless-Type MMTV Integration Site Family.

In some embodiments, the microsphere composition comprises about 1 ng to about 100 mg of one or more cytokines. For example, about 100 mg of one or more cytokines, about 90 mg of one or more cytokines, about 80 mg of one or more cytokines, about 70 mg of one or more cytokines, about 60 mg of one or more cytokines, about 50 mg of one or more cytokines, about 40 mg of one or more cytokines, about 30 mg of one or more cytokines, about 20 mg of one or more cytokines, about 10 mg of one or more cytokines, about 9 mg of one or more cytokines, about 8 mg of one or more cytokines, about 7 mg of one or more cytokines, about 6 mg of one or more cytokines, about 5 mg of one or more cytokines, about 4 mg of one or more cytokines, about 3 mg of one or more cytokines, about 2 mg of one or more cytokines, about 1 mg of one or more cytokines, about 0.9 mg of one or more cytokines, about 0.8 mg of one or more cytokines, about 0.7 mg of one or more cytokines, about 0.6 mg of one or more cytokines, about 0.5 mg of one or more cytokines, about 0.4 mg of one or more cytokines, about 0.3 mg of one or more cytokines, about 0.2 mg of one or more cytokines, about 0.1 mg of one or more cytokines, about 0.09 mg of one or more cytokines, about 0.08 mg of one or more cytokines, about 0.07 mg of one or more cytokines, about 0.06 mg of one or more cytokines, about 0.05 mg of one or more cytokines, about 0.04 mg of one or more cytokines, about 0.03 mg of one or more cytokines, about 0.02 mg of one or more cytokines, or about 0.01 mg of one or more cytokines. For example, about 1000 ng of one or more cytokines, about 900 ng of one or more cytokines, about 800 ng of one or more cytokines, about 700 ng of one or more cytokines, about 600 ng of one or more cytokines, about 500 ng of one or more cytokines, about 400 ng of one or more cytokines, about 300 ng of one or more cytokines, about 200 ng of one or more cytokines. In some embodiments, the microsphere composition comprises about 1 ng to about 100 ng of one or more cytokines. For example, about 100 ng of one or more cytokines, about 90 ng of one or more cytokines, about 80 ng of one or more cytokines, about 70 ng of one or more cytokines, about 60 ng of one or more cytokines, about 50 ng of one or more cytokines, about 40 ng of one or more cytokines, about 30 ng of one or more cytokines, about 20 ng of one or more cytokines, about 10 ng of one or more cytokines, about 9 ng of one or more cytokines, about 8 ng of one or more cytokines, about 7 ng of one or more cytokines, about 6 ng of one or more cytokines, about 5 ng of one or more cytokines, about 4 ng of one or more cytokines, about 3 ng of one or more cytokines, about 2 ng of one or more cytokines, or about 1 ng of one or more cytokines.

In some embodiments of the method, the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CDS, CD8B, CD5L, CD68, CD55, CD7, CD73, CD58, CD74, CD80, CD79, CD84, CD93, CD99, CD164, and CD40L.

In some embodiments, the microsphere composition comprises about 1 ng to about 100 mg of one or more CD antigens. For example, about 100 mg of one or more CD antigens, about 90 mg of one or more CD antigens, about 80 mg of one or more CD antigens, about 70 mg of one or more CD antigens, about 60 mg of one or more CD antigens, about 50 mg of one or more CD antigens, about 40 mg of one or more CD antigens, about 30 mg of one or more CD antigens, about 20 mg of one or more CD antigens, about 10 mg of one or more CD antigens, about 9 mg of one or more CD antigens, about 8 mg of one or more CD antigens, about 7 mg of one or more CD antigens, about 6 mg of one or more CD antigens, about 5 mg of one or more CD antigens, about 4 mg of one or more CD antigens, about 3 mg of one or more CD antigens, about 2 mg of one or more CD antigens, about 1 mg of one or more CD antigens, about 0.9 mg of one or more CD antigens, about 0.8 mg of one or more CD antigens, about 0.7 mg of one or more CD antigens, about 0.6 mg of one or more CD antigens, about 0.5 mg of one or more CD antigens, about 0.4 mg of one or more CD antigens, about 0.3 mg of one or more CD antigens, about 0.2 mg of one or more CD antigens, about 0.1 mg of one or more CD antigens, about 0.09 mg of one or more CD antigens, about 0.08 mg of one or more CD antigens, about 0.07 mg of one or more CD antigens, about 0.06 mg of one or more CD antigens, about 0.05 mg of one or more CD antigens, about 0.04 mg of one or more CD antigens, about 0.03 mg of one or more CD antigens, about 0.02 mg of one or more CD antigens, or about 0.01 mg of one or more CD antigens. For example, about 1000 ng of one or more CD antigens, about 900 ng of one or more CD antigens, about 800 ng of one or more CD antigens, about 700 ng of one or more CD antigens, about 600 ng of one or more CD antigens, about 500 ng of one or more CD antigens, about 400 ng of one or more CD antigens, about 300 ng of one or more CD antigens, about 200 ng of one or more CD antigens. In some embodiments, the microsphere composition comprises about 1 ng to about 100 ng of one or more CD antigens. For example, about 100 ng of one or more CD antigens, about 90 ng of one or more CD antigens, about 80 ng of one or more CD antigens, about 70 ng of one or more CD antigens, about 60 ng of one or more CD antigens, about 50 ng of one or more CD antigens, about 40 ng of one or more CD antigens, about 30 ng of one or more CD antigens, about 20 ng of one or more CD antigens, about 10 ng of one or more CD antigens, about 9 ng of one or more CD antigens, about 8 ng of one or more CD antigens, about 7 ng of one or more CD antigens, about 6 ng of one or more CD antigens, about 5 ng of one or more CD antigens, about 4 ng of one or more CD antigens, about 3 ng of one or more CD antigens, about 2 ng of one or more CD antigens, or about 1 ng of one or more CD antigens. In some embodiments of the method, the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, and Pleiotrophin.

In some embodiments, the microsphere composition comprises about 1 ng to about 100 mg of one or more neurotrophins. For example, about 100 mg of one or more neurotrophins, about 90 mg of one or more neurotrophins, about 80 mg of one or more neurotrophins, about 70 mg of one or more neurotrophins, about 60 mg of one or more neurotrophins, about 50 mg of one or more neurotrophins, about 40 mg of one or more neurotrophins, about 30 mg of one or more neurotrophins, about 20 mg of one or more neurotrophins, about 10 mg of one or more neurotrophins, about 9 mg of one or more neurotrophins, about 8 mg of one or more neurotrophins, about 7 mg of one or more neurotrophins, about 6 mg of one or more neurotrophins, about 5 mg of one or more neurotrophins, about 4 mg of one or more neurotrophins, about 3 mg of one or more neurotrophins, about 2 mg of one or more neurotrophins, about 1 mg of one or more neurotrophins, about 0.9 mg of one or more neurotrophins, about 0.8 mg of one or more neurotrophins, about 0.7 mg of one or more neurotrophins, about 0.6 mg of one or more neurotrophins, about 0.5 mg of one or more neurotrophins, about 0.4 mg of one or more neurotrophins, about 0.3 mg of one or more neurotrophins, about 0.2 mg of one or more neurotrophins, about 0.1 mg of one or more neurotrophins, about 0.09 mg of one or more neurotrophins, about 0.08 mg of one or more neurotrophins, about 0.07 mg of one or more neurotrophins, about 0.06 mg of one or more neurotrophins, about 0.05 mg of one or more neurotrophins, about 0.04 mg of one or more neurotrophins, about 0.03 mg of one or more neurotrophins, about 0.02 mg of one or more neurotrophins, or about 0.01 mg of one or more neurotrophins. For example, about 1000 ng of one or more neurotrophins, about 900 ng of one or more neurotrophins, about 800 ng of one or more neurotrophins, about 700 ng of one or more neurotrophins, about 600 ng of one or more neurotrophins, about 500 ng of one or more neurotrophins, about 400 ng of one or more neurotrophins, about 300 ng of one or more neurotrophins, about 200 ng of one or more neurotrophins. In some embodiments, the microsphere composition comprises about 1 ng to about 100 ng of one or more neurotrophins. For example, about 100 ng of one or more neurotrophins, about 90 ng of one or more neurotrophins, about 80 ng of one or more neurotrophins, about 70 ng of one or more neurotrophins, about 60 ng of one or more neurotrophins, about 50 ng of one or more neurotrophins, about 40 ng of one or more neurotrophins, about 30 ng of one or more neurotrophins, about 20 ng of one or more neurotrophins, about 10 ng of one or more neurotrophins, about 9 ng of one or more neurotrophins, about 8 ng of one or more neurotrophins, about 7 ng of one or more neurotrophins, about 6 ng of one or more neurotrophins, about 5 ng of one or more neurotrophins, about 4 ng of one or more neurotrophins, about 3 ng of one or more neurotrophins, about 2 ng of one or more neurotrophins, or about 1 ng of one or more neurotrophins. In some embodiments of the method, the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

In some embodiments, the microsphere composition comprises about 0.1 mg to about 300 mg of one or more miRNAs. For example, about 300 mg of one or more miRNAs, about 275 mg of one or more miRNAs, about 250 mg of one or more miRNAs, about 225 mg of one or more miRNAs, about 200 mg of one or more miRNAs, about 175 mg of one or more miRNAs, about 150 mg of one or more miRNAs, about 125 mg of one or more miRNAs, about 100 mg of one or more miRNAs, about 90 mg of one or more miRNAs, about 80 mg of one or more miRNAs, about 75 mg of one or more miRNAs, about 70 mg of one or more miRNAs, about 60 mg of one or more miRNAs, about 50 mg of one or more miRNAs, about 40 mg of one or more miRNAs, about 30 mg of one or more miRNAs, about 20 mg of one or more miRNAs, about 10 mg of one or more miRNAs, about 9 mg of one or more miRNAs, about 8 mg of one or more miRNAs, about 7 mg of one or more miRNAs, about 6 mg of one or more miRNAs, about 5 mg of one or more miRNAs, about 4 mg of one or more miRNAs, about 3 mg of one or more miRNAs, about 2 mg of one or more miRNAs, about 1 mg of one or more miRNAs, about 0.9 mg of one or more miRNAs, about 0.8 mg of one or more miRNAs, about 0.7 mg of one or more miRNAs, about 0.6 mg of one or more miRNAs, about 0.5 mg of one or more miRNAs, about 0.4 mg of one or more miRNAs, about 0.3 mg of one or more miRNAs, about 0.2 mg of one or more miRNAs, or about 0.1 mg of one or more miRNAs. For example, about 1000 ng of one or more miRNAs, about 900 ng of one or more miRNAs, about 800 ng of one or more miRNAs, about 700 ng of one or more miRNAs, about 600 ng of one or more miRNAs, about 500 ng of one or more miRNAs, about 400 ng of one or more miRNAs, about 300 ng of one or more miRNAs, about 200 ng of one or more miRNAs.

In some embodiments, the microsphere composition comprises about 0.01 mg/kg to about 5 mg/kg of one or more of microRNAs. For example, about 5 mg/kg of one or more of microRNAs, about 4 mg/kg of one or more microRNAs, about 3 mg/kg of one or more of microRNAs, about 2 mg/kg of one or more microRNAs, about 1 mg/kg of one or more microRNAs, about 0.9 mg/kg of one or more microRNAs, about 0.8 mg/kg of one or more microRNAs, about 0.7 mg/kg of one or more microRNAs, about 0.6 mg/kg of one or more microRNAs, about 0.5 mg/kg of one or more microRNAs, about 0.4 mg/kg of one or more microRNAs, about 0.3 mg/kg of one or more microRNAs, about 0.2 mg/kg of one or more microRNAs, about 0.1 mg/kg of one or more microRNAs, about 0.09 mg/kg of one or more microRNAs, about 0.08 mg/kg of one or more microRNAs, about 0.07 mg/kg of one or more microRNAs, about 0.06 mg/kg of one or more microRNAs, about 0.05 mg/kg of one or more microRNAs, about 0.04 mg/kg of one or more microRNAs, about 0.03 mg/kg of one or more microRNAs, about 0.02 mg/kg of one or more microRNAs, or about 0.01 mg/kg of one or more microRNAs.

As used herein a “buffer” refers to a biological buffer that can maintain a constant pH over a given range by neutralizing the effects of hydrogen ions. A buffer can be used to maintain or control the acidity of a solution within a desired physiological range. The pH has an effect on the structure and function of proteins, enzymatic reactions and cellular metabolism, thus buffers significantly contribute to the outcome of such studies. Buffers typically have pKa between 6.0 and 8.0, a high water solubility and low organic solvent solubility, a low lipid solubility with biological membrane impermeability, a minimal effect on the dissociation of the buffer from any changes to temperature, ionic strength and concentration, a high stability with resistance to enzymatic degradation, does not absorb light in the visible or UV spectrum, and has minimal interactions with salt and other reaction constituents. In some embodiments of the method, the buffer is selected from: Acetate buffers, ACES, ADA, AMP, AMPSO, AMPD, BES, Bicine, Bis-Tris, Bis-Tris Propane, CABS, CAPSO, CAPS, CHES, Citrate buffers, DIPSO, EPPS, Gly-Gly, HEPBS, HEPES, HEPPSO, PBS, PIPES, POPSO, MES, MOBS, MOPSO, MOPS, Sodium carbonate buffers, Sodium bicarbonate buffers, TABS, TAPS, TAPSO, TBS, TEA, TES, Tricine, and Tris.

In some embodiments of the method, the carrier protein is selected from bovine serum albumin, human serum albumin, equine serum albumin, goat serum albumin, porcine serum albumin, rat serum albumin, mouse serum albumin, chicken serum albumin, and chicken white albumin.

In some embodiments of the method, the salt of the bulk aqueous solution is selected from: chloride salts (e.g., NaCl), fluoride salts, phosphate salts, iron salts, carbonate salts, bicarbonate salts, sulfate salts, bisulfate salts, and potassium dichromate.

As used herein, the term “surfactant” refers to compounds that lower the surface tension (or interfacial tension) between two liquids, or between a liquid and a solid. Surfactants may act as detergents, wetting agents, emulsifiers, foaming agents, or dispersants. In certain embodiments of the method, non-ionic surfactants can be used. In certain embodiments of the method, anionic surfactants can be used. In certain embodiments of the method, cationic surfactants can be used. In some embodiments of the method, the aqueous surfactant of the bulk aqueous solution is selected from: poly vinyl alcohol (PVA), polysorbate (e.g. Tweens), sorbitan esters (SPAN), poly(vinyl pyrrolidone) (PVP; also known as povidone, and poloxamers).

In some embodiments of the method, the alcohol of the bulk aqueous solution is selected from: ethanol, propanol, n-butanol, 1-pentanol, and structural and functional isomers thereof.

In some embodiments of the method, the method further comprises lyophilizing the isolated microspheres.

In some embodiments of the method, the method further comprising sieving the isolated microspheres and collecting the microspheres that are between about 10 μm to about 20 μm in diameter.

In some embodiments of the method, the ratio of the polar solvent to the aqueous surfactant of the secondary emulsion is about 1:20 to about 1:1.

### EXAMPLES

### Materials and Methods

### SASC Fabrication

Different microsphere batches for each growth factor were made using a modified double (w/o/w) emulsion solvent evaporation method^{29,30}. Briefly, 250 mg of PLGA was dissolved in 4 mL of dichloromethane (DCM). Bulk aqueous solution of 1% (w/v) PVA+1% (v/v) IPA+10% (w/v) NaCl was prepared. In a 250 mL beaker, 100 mL of 2 (v/v) % IPA was added to 100 mL of a 2 (w/v) % PVA solution. NaCl was added to make a 10% w/v bulk solution. Growth factors were then diluted with PBS+4% BSA to reach a final concentration of 1% BSA for HGH and FGF-18 and 3% for TGF-β1 and a final volume of 100 μL. The theoretical loading of each growth factor was 10 ng growth factor/mg PLGA. Blank microspheres were prepared using 100 μL of PBS.

After PLGA dissolution, the growth factor or blank solutions were emulsified by vortexing at max speed for 30 seconds. Immediately, 4 mL of 5% Polyvinyl alcohol (PVA) was added to make the secondary emulsion and this was vortexed for another 30 seconds at max speed. The double emulsion was injected into the bulk solution through an 18 G needle. The solution was stirred at 600 RPM for 3 hours to allow for DCM extraction and evaporation. After evaporation, microspheres were centrifuged at 2000 rcf for 3 minutes, subsequently washed with 250 mL water 3 times by filtration and lyophilized overnight. Microspheres in the 10-20 μm size range were separated by sieving and stored at −20° C. with desiccant until combined to form the final SASC composition. The composition of SASC used for the following studies was 20% by weight FGF-18 microspheres, 20% by weight TGF-β1 microspheres, 20% by weight IGF-1 microspheres, 20% by weight HGH microspheres and remaining blank microspheres. The blank microspheres were added to this composition to allow for increases in weight composition to allow for addition of an additional factor if needed. SASC and blank microspheres were sterilized for 20 minutes.

### Particle Size Distribution

Microspheres size distribution was determined by sieving. Mass fractions were determined by dividing the weight of microspheres in the respective size bucket by the total weight of microspheres determined by the sum in all sieves. Microspheres in the 10-20 μm range were separated and sterilized for 20 minutes. Sterilized microspheres were used for further in vitro and in vivo testing.

### Scanning Electron Microscopy

For each growth factor loaded microsphere batch, sieved microspheres were mounted on carbon tape and sputter coated (Denton Vacuum Desk V) at 20 mA for 60 seconds. SEM (JEOL JSM-5900LV) images were taken using 10 kV voltage and spot size 23. 1,000× magnification was used to see general shape and size distribution and 10,000× magnification to see surface smoothness and any porosity. As the electron beam at 10,000× magnification damaged the microsphere structure, brightness and contrast were adjusted on a single sphere and images were captured using an adjacent sphere.

### Loading Efficiency

For each growth factor, 10 mg microspheres were dissolved in 2 mL DCM at 37° C. for one hour (n=4). PBS, 2 mL, was added and the solution was vortexed for 30 seconds to allow extraction of the growth factor followed by centrifugation at 2000 rcf for 3 minutes for phase separation. The top layer was aspirated and replaced with fresh PBS to repeat the process two times for a final extract volume of 6 mL. ELISA was performed in triplicate to calculate the loading efficiency using 100 ng as the theoretical mass of growth factor loaded in 10 mg microspheres.

### Control Release Study

Release profiles for each growth factor were made for 10 mg microspheres in 2 mL PBS (n=4). At pre-determined time points, samples were centrifuged for 1 minute at 2000 rcf and 1 mL of the medium was collected for ELISA and replaced by 1 mL fresh PBS. The sample was briefly vortexed for microsphere resuspension and placed back into the water bath. ELISA was performed in triplicate to calculate the released mass and % release was calculated using the loading efficiency.

### ADSC Isolation and Culture

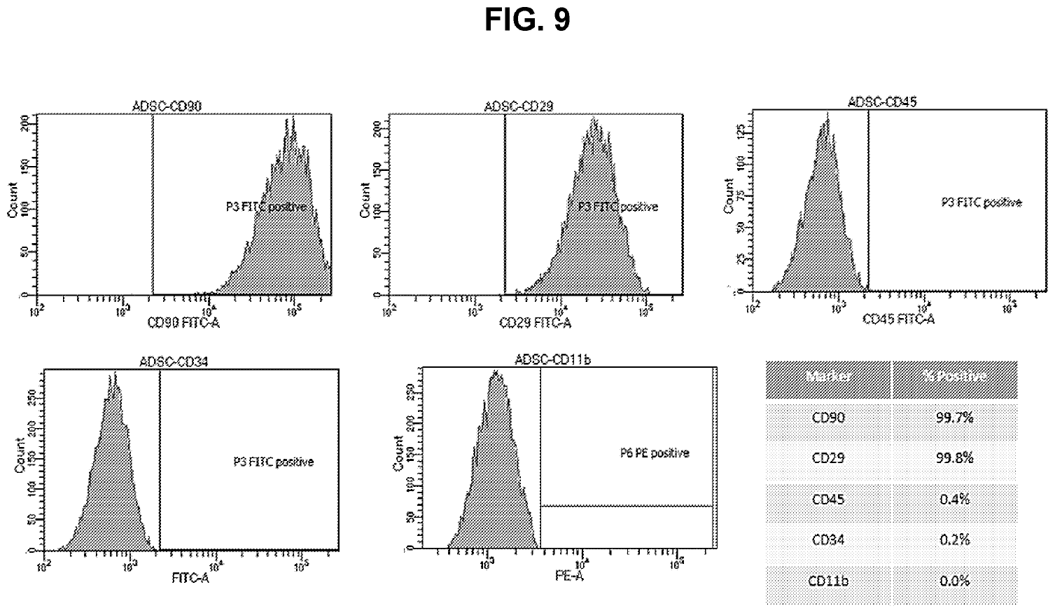

ADSCs were isolated from 6-8 week-old Sprague Dawley (SD) rats in accordance with the experimental guidelines and regulations approved by University of Connecticut Health Center Institutional Animal Care and Use Committee (IACUC) approved protocol. ADSCs were isolated and purified as described by Bhattacharjee et al⁵¹. Rats were euthanized with CO₂ asphyxiation followed by cervical dislocation. Euthanized rats were weighed, shaved and cleaned with 70% Ethanol. Inguinal fat pads were isolated and weighed. The fat pads were collected, washed in sterile Hank's Balanced Salt Solution (HBSS) with 1% penn/strep and minced into small pieces. The pieces were added to 25 mL collagenase type I (300U/mL) (Invitrogen) in HBSS and agitated at 37° C. for 90 min⁵¹. The resulting cell suspension was filtered through a 70 μm filter (BD Bioscience) for the removal of the solid aggregates. An equal volume of DMEM-F12 with 10% FBS and 1% penn/strep was added to neutralize the collagenase. The mixture was centrifuged at 1500 rpm for 10 min, the supernatant was discarded and the pellet was re-suspended in 2 mL of red cell lysis buffer and incubated for 2 min at 37° C. The mixture was again centrifuged at 1500 rpm for 10 min. The supernatant was discarded and cells were counted and plated in a T150 flask containing DMEM-F12 with 10% FBS and 1% penn/strep⁵¹. The media was changed after 24-48 hours to remove cell debris and non-adhering cells. PO ADSCs were cultured in T150 flasks for 2 weeks, in DMEM-F12 supplemented with 1% penicillin/streptomycin. ADSCs reaching 80-90% confluence were detached with 0.25% trypsin (Thermo Scientific) at 37° C., centrifuged at 1500 rpm for 10 minutes and re-plated. ADSCs were characterized by flow cytometry using cell surface markers (Becton-Dickinson LSR II, BD Biosciences, USA) at passage 3 (P3). Cells were washed using sterile PBS, centrifuged and re-suspended in sterile Fluorescence-Activated Cell Sorting (FACS) buffer (PBS, 1% FBS) containing 10 μL of the FITC-conjugated CD29 antibody, Fluorescein isothiocyanate (FITC) conjugated CD90 antibody, FITC conjugated CD34, FITC conjugated CD45, and PE conjugated CD11b for 30 min. Unlabeled cells were used as controls⁵¹. Cells were then scanned with FACS; acquired and gated using forward scatter (FSC) and side scatter (SSC) parameters to exclude cell debris and aggregates (See FIG. 9).

### Co-Culture

All incubation and culturing was done at 37° C., 5% CO₂ and 85% RH. Primary chondrocytes were passaged through P4 and then 100,000 cells were seeded into a 24 well plate for Nitric Oxide and PCR analysis. Media containing DMEM-F12, 10% FBS and 1% penn/strep was used to passage and initially seeding the cells. After cell attachment, the media was changed to DMEM-F12, 5% FBS and 1% penn/strep for the negative control and DMEM-F12, 5% FBS, 1% penn/strep and 20 ng/mL IL-1β for all other groups. Cells were incubated for 24 hours to allow induction of inflammation. After induction, 100,000 ADSCs, blank microspheres or SASC were added into respective transwell inserts for co-culture with inflamed chondrocytes. The media was also refreshed with fresh media (DMEM-F12+5% FBS+1% penn/strep±20 ng/mL IL-1β).

The treatment went on for 72 hours after which the media was collected for nitric oxide analysis and assay.

Nitric oxide concentration was measured using the Griess reagent assay. Standards were made by serial dilution of the stock in 5% DMEM-F12+5% FBS+1% penn/strep. Standards and samples were then plates according to the manufacturer's procedures. The plate was incubated in for 30 minutes and the absorbance was read at 550 nm. A standard curve was made from blank subtracted standards and then nitric oxide content was calculated from the blank subtracted sample readings using the best-fit linear trend line.

### Quantitative Real Time-PCR

qRT-PCR was done using a procedure used in our group⁵¹. Briefly, total RNA was isolated from cells pooled from 4 wells using the RNeasy Mini Kit (Qiagen; Ref 74104) according to the manufacturer's instructions. For cDNA synthesis, 2-4 μg total RNA was used as a template for cDNA synthesis using EcoDry premix (Takara Cat. 639549) in a total volume of 20 μL⁵¹. For quantitative real-time PCR, iCycler Thermal Cycler Base (Bio-Rad) and iQ Supermix (Bio-Rad), SOX-9, ADAMTS5, PRG4, and GAPDH Taqman gene probes were used. The threshold cycle values of target genes were standardized against GAPDH expression and normalized to the expression in the untreated control culture. The fold change in expression of sample triplicates was calculated using the AACt comparative threshold cycle method⁵¹.

### Induction and Treatment of Osteoarthritis

This experimental protocol was approved by the Institutional Animal Care Committee, UConn Health. Rats Sprague Dawley rats were acclimated for at least 24 hours before procedure after being purchased from the vendor. On day 0 and day 3, animals were injected with 500 U of Collagenase type II to induce an OA phenotype. Animals were then divided into 4 treatment groups (n=11) injected on day 7: Osteoarthritis Control treated with Saline; Rats injected with 1 million ADSCs suspended in saline; Rats injected with 1 million SASC particles suspended in saline; Rats injected with 1 million blank microspheres in saline. A second treatment dose was given to all animals at 5 weeks after first treatment. Using a hemacytometer, it was determined that there were 150,000 spheres/mg and used this to determine the corresponding mass needed for 1 million microspheres (6.67 mg). For the SASC group, the dose of each growth factor was then determined as 9.256 ng HGH and FGF-18, 11.86 ng IGF-1 and 5.424 ng TGF-β1.

On each day of injection, rats were anesthetized with 2-4% isofluorane. The right knee was shaved and the diameter of both knees were measured by caliper. The right knee was prepped with Betadine and 70% ethanol and under fluoroscopic guidance, the respective solution was intra-articularly injected. The extent of knee swelling was monitored weekly until sacrifice at week 9.

### Histology

Upon sacrifice, knee joints were harvested and separated for histological and biomechanical evaluation. Contralateral joints were taken as a healthy control. Those joints taken for biomechanics were wrapped in PBS dampened gauze, vacuum sealed and then stored in −20° C. until analysis. Joints used for histology were tied in a fully extended position on a cotton applicator stick and then fixed in 10% formalin for 5-7 days at 4° C. and then washed 3 times with PBS in 1 hour storage intervals at 4° C. Decalcification was done in Cal-EX for 3 days at 4° C., changing the solution every day followed by rinsing overnight with constantly running distilled water. Joints were then cut in half along the frontal plane and submitted to a core facility for processing and paraffin embedding. 5 μm sections were taken at about 350 μm into the block and stained with H&E followed by a 15 minute incubation in Safranin O prior to dehydration. Articular surface areas of the femoral condyles and tibial plateaus (areas stained red with safranin O), measured between the ligament insertion site and the lateral or medial end of the bone were identified and areas were quantified using ImageJ image analysis software. Areas of degeneration where there was no red staining by safranin O were measured using imageJ and the % degeneration at the joint was measured using the following formula:

$$\% Degeneration = \frac{Σ Degenerated Areas}{Σ Theoretical Healthy Articular Surface Area} * 100$$

### Biomechanics

Joints were thawed in PBS with low agitation for at least 30 minutes. The joint was then dissected to expose the femoral and tibial articular cartilage. For the automated indentation of the articular cartilage, a 70-N multiaxial load cell with an amplification module was employed and calibrated prior to each use. The femurs and tibias in PBS were subjected to automated indentation at 12 and 22 predefined positions, respectively, using a 0.3 mm spherical tip indenter over each surface. Positions were evenly mapped on the lateral and medial sides of each bone. Automated indentation was done to a depth of 0.05 mm, at a speed of 0.05 mm/s with 10 seconds relaxation, as an initial scan and to ensure sufficient signal (load) was obtained with a 100 Hz data acquisition rate. Stress relaxation curves were observed with indentation of the articular cartilage. Structural stiffness was then calculated at an appropriate indentation depth and mapped at the indented positions.

### Statistics

Minitab 19 was used to run an ANOVA with a two-sided 95% confidence interval for each in vitro comparison and degenerated area comparison. To analyze biomechanical differences between samples, a linear mixed effects model with two-sided 95% confidence interval was done in minitab 19 to account for multiple measurement positions being combined to make up the whole cartilage surface stiffness. A Tukey's means comparison was done to evaluate inter-group differences. Graphpad prism 6 was used to graph all figure data.

### Fabrication and Characterization of the SASC System

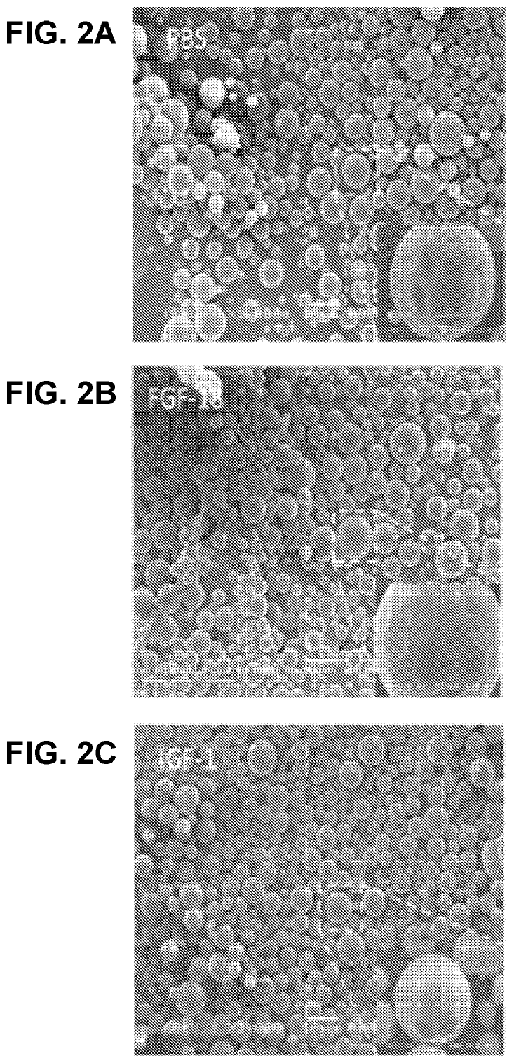

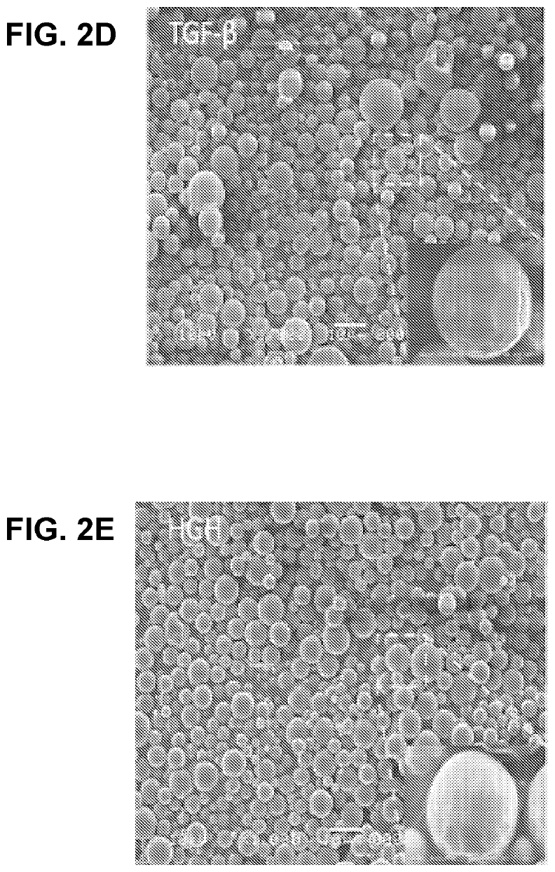

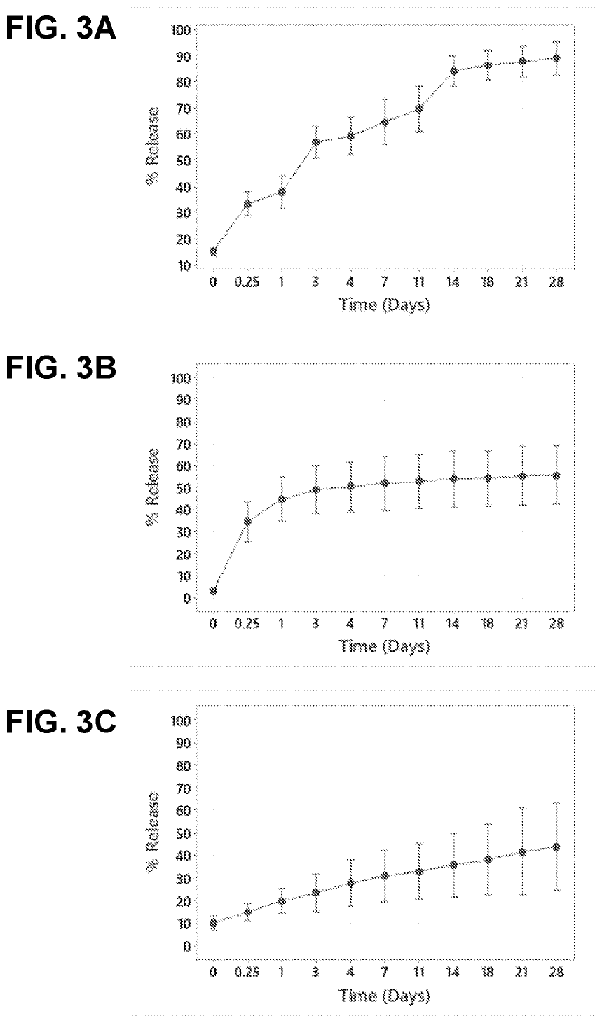

Growth factors, FGF-18, IGF-1, HGH and TGF-β1, were loaded individually into the PLGA matrix (10 ng growth factor/mg PLGA) using a modified double emulsion method^{29,30}. Scanning electron microscopy (SEM) was used to visualize microspheres that were in the 10-20 μm range as collected by sieving. 1000× magnification was used to see shape and size while 10,000× magnification was used to see surface smoothness of each microsphere batch (FIG. 2). Microspheres of all batches, irrespective of growth factor loaded, maintained a smooth surface and similar size distribution while shrinking and surface wrinkling observed in the higher magnification images was an artifact of the electron beam. Loading efficiency of IGF-1 was 88.95±9.66% (Table 1). Release was observed separated into three phases: an initial burst with 30% surface loaded protein released within 1 day, a diffusion controlled phase over the next 10 days and lastly an equilibrium controlled release until 90% was released by the 28th day (FIG. 3A). The loading efficiency of HGH and TGF-β1 was found to be low due the large size, hence bovine serum albumin (BSA) was used as a carrier protein to enhance the respective loading efficiencies. HGH with 1% BSA achieved 69.42±12.48% loading (Table 1). 50% of the loaded HGH was released over the first day and plateaued at this level for the remainder of the 28 day release period (FIG. 3B). TGF-β1 was loaded at 40.68±4.04% efficiency with 3% BSA used (Table 1). The release of this large, hydrophobic molecule showed minimal burst and a much slower, linear release profile in which about 50% was released over 28 days (FIG. 3C).

TABLE 1
Loading Efficiency of IGF-1, HGH and TGF-β1 Microspheres
(n = 4). Growth factors used different concentrations of carrier
protein to achieve loading depending on their physical properties.

| Growth Factor | % BSA | Loading Efficiency |
|---|---|---|
| IGF-1 | 0% | 88.95 ± 9.66% |
| HGH | 1% | 69.42 ± 12.48% |
| TGF-β1 | 3% | 40.68 ± 4.04% |

### Adipose Derived Stem Cell (ADSC) Characterization by Flow Cytometry

Isolated ADSCs were characterized by flow cytometry using cell surface markers (Becton-Dickinson LSR II, BD Biosciences, USA) at passage 3 (P3). Cells were washed using sterile PBS, centrifuged and re-suspended in sterile Fluorescence-Activated Cell Sorting (FACS) buffer (PBS, 1% FBS) containing 10 μL of the FITC-conjugated CD29 antibody, Fluorescein isothiocyanate (FITC) conjugated CD90 antibody, FITC conjugated CD34, FITC conjugated CD45, and PE conjugated CD11b for 30 min. Unlabeled cells were used as controls. Cells were then scanned with FACS; acquired and gated using forward scatter (FSC) and side scatter (SSC) parameters to exclude cell debris and aggregates.

### Example 1

### Investigating the Anti-Inflammatory and Chondro-Protective Effects of SASC

A co-culture system was used as an indirect in vitro osteoarthritis model (FIG. 1A). Healthy chondrocytes and chondrocytes treated with a pro-inflammatory cytokine IL-1β (a prominent inflammatory marker produced during early stages of OA) were used as respective sham and negative treatment controls. The remaining cells were divided into 3 groups: Inflamed chondrocytes treated with ADSCs, Inflamed chondrocytes treated with blank (PBS loaded particles); Inflamed chondrocytes treated with synthetic artificial stem cells (SASC). The approximate dose of each growth factor in the SASC group was 920 pg HGH and FGF-18, 1186 pg IGF-1 and 550 pg TGF-β1.

### Nitric Oxide Assay Evaluating the Anti-Inflammatory Property of SASC

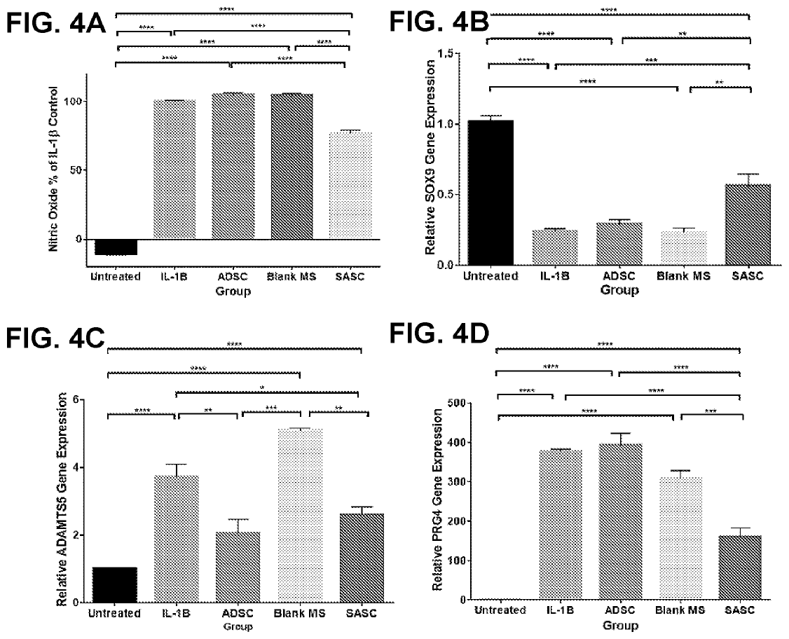

Nitric oxide (NO) is expressed in the chondrocyte inflammatory process. An in vitro co-culture system of chondrocytes and ADSCs was used to investigate the production of nitric oxide. Chondrocytes upon treatment with IL-1β showed significant increase in NO production that indicates inflammation in chondrocytes. The addition of ADSCs and the blank group did not reduce NO production of inflamed chondrocytes. However, the addition of SASC showed significantly lower production of NO, indicating the anti-inflammatory property of SASC (FIG. 4A).

### Gene Expression Analysis Evaluating the Anti-Inflammatory and Chondro Protective Effect

To investigate the inhibition of catabolic responses of IL-1β by SASC, quantitative real-time PCR (qRT-PCR) was performed to determine the gene expression of early chondrogenic transcription factor SOX9, matrix-degrading enzyme, a disintegrin and metalloproteinase with thrombospondin motifs 5 (ADAMTS5) as well as anti-inflammatory factor proteoglycan 4 (PRG4), also known as lubricin. The IL-1β treated group showed significant upregulation of ADAMTSS (FIG. 4C), while a significant downregulation was observed for SOX9 (FIG. 4B) and PRG4 (FIG. 4D) compared to healthy chondrocytes. ADSCs were found to only attenuate the increased mRNA level of ADAMTS5 over the three day treatment period. SASC showed significant upregulation of SOX9 and attenuation of increased NO, PRG4 and mRNA level of ADAMTS5 (FIGS. 4B-4D), indicating the significant anti-inflammatory and chondroprotective effect of SASC and versatility of a synthetic artificial stem cell (SASC) treatment compared to ADSCs.

TABLE 2
Nitric Oxide % of IL-1β ANOVA Summary

| Source | DF | Adj SS | Adj MS | F-Value | P-Value |
|---|---|---|---|---|---|
| Group | 4 | 533932 | 133483 | 974.32 | 0 |
| Error | 251 | 34387 | 137 | | |
| Total | 255 | 568319 | | | |

| Grouping Information | | | | | |
|---|---|---|---|---|---|
| Using the Tukey Method | | | | | |
| and 95% Confidence | | | | | |
| Group | N | Mean | Grouping | | |
| ADSC | 47 | 104.69 | A | | |
| Blank MS | 23 | 104.12 | A | | |
| IL-1B | 66 | 100 | A | | |
| SASC | 60 | 76.64 | | B | |
| Untreated | 60 | −10.474 | | | C |
| Means that do not share | | | | | |
| a letter are significantly | | | | | |
| different. | | | | | |

| | Difference | SE of | | | Adjusted |
|---|---|---|---|---|---|
| Difference of Levels | of Means | Difference | 95% CI | T-Value | P-Value |
| Blank MS − ADSC | −0.57 | 2.98 | (−8.70, 7.56) | −0.19 | 1 |
| IL-1B − ADSC | −4.69 | 2.23 | (−10.79, 1.41) | −2.1 | 0.22 |
| SASC − ADSC | −28.05 | 2.28 | (−34.28, −21.83) | −12.3 | 0 |
| Untreated − ADSC | −115.16 | 2.28 | (−121.39, −108.94) | −50.51 | 0 |
| IL-1B − Blank MS | −4.12 | 2.83 | (−11.85, 3.62) | −1.45 | 0.593 |
| SASC − Blank MS | −27.48 | 2.87 | (−35.32, −19.65) | −9.57 | 0 |
| Untreated − Blank MS | −114.59 | 2.87 | (−122.43, −106.76) | −39.92 | 0 |
| SASC − IL-1B | −23.36 | 2.09 | (−29.06, −17.67) | −11.19 | 0 |
| Untreated − IL-1B | −110.47 | 2.09 | (−116.17, −104.78) | −52.91 | 0 |
| Untreated − SASC | −87.11 | 2.14 | (−92.94, −81.28) | −40.76 | 0 |
| Individual confidence | | | | | |
| level = 99.32% | | | | | |
| Tukey Simultaneous | | | | | |
| 95% CIs | | | | | |

TABLE 3
SOX9 Fold Change ANOVA Summary

| Source | DF | Adj SS | Adj MS | F-Value | P-Value |
|---|---|---|---|---|---|
| Group | 4 | 6.3515 | 1.58786 | 133.93 | 0 |
| Error | 55 | 0.6521 | 0.01186 | | |
| Total | 59 | 7.0035 | | | |

Tukey Pairwise Comparisons

| Grouping Information | | | | | |
|---|---|---|---|---|---|
| Using the Tukey Method | | | | | |
| and 95% Confidence | | | | | |
| Group | N | Mean | Grouping | | |
| Untreated | 15 | 1.0148 | A | | |
| SASC | 12 | 0.4038 | | B | |
| IL-1B | 15 | 0.2384 | | | C |
| Blank MS | 9 | 0.2312 | | | C |
| ADSC | 9 | 0.227 | | | C |
| Means that do not share | | | | | |
| a letter are significantly | | | | | |
| different. | | | | | |

Tukey Simultaneous Tests for Differences of Means

| | Difference | SE of | | | Adjusted |
|---|---|---|---|---|---|
| Difference of Levels | of Means | Difference | 95% CI | T-Value | P-Value |
| Blank MS − ADSC | 0.0042 | 0.0513 | (−0.1406, 0.1490) | 0.08 | 1 |
| IL-1B − ADSC | 0.0113 | 0.0459 | (−0.1182, 0.1408) | 0.25 | 0.999 |
| SASC − ADSC | 0.1768 | 0.048 | (0.0413, 0.3122) | 3.68 | 0.005 |
| Untreated − ADSC | 0.7878 | 0.0459 | (0.6583, 0.9173) | 17.16 | 0 |
| IL-1B − Blank MS | 0.0071 | 0.0459 | (−0.1224, 0.1367) | 0.16 | 1 |
| SASC − Blank MS | 0.1726 | 0.048 | (0.0371, 0.3081) | 3.59 | 0.006 |
| Untreated − Blank MS | 0.7836 | 0.0459 | (0.6541, 0.9132) | 17.07 | 0 |
| SASC − IL-1B | 0.1654 | 0.0422 | (0.0465, 0.2844) | 3.92 | 0.002 |
| Untreated − IL-1B | 0.7765 | 0.0398 | (0.6643, 0.8886) | 19.53 | 0 |
| Untreated − SASC | 0.611 | 0.0422 | (0.4920, 0.7300) | 14.49 | 0 |
| Individual confidence | | | | | |
| level = 99.34% | | | | | |
| Tukey Simultaneous | | | | | |
| 95% CIs | | | | | |

TABLE 4
ADAMTS5 Fold Change ANOVA Summary.

| Source | DF | Adj SS | Adj MS | F-Value | P-Value |
|---|---|---|---|---|---|
| Group | 4 | 77.8 | 19.4493 | 19.49 | 0 |
| Error | 51 | 50.89 | 0.9978 | | |
| Total | 55 | 128.69 | | | |

Tukey Pairwise Comparisons

| Grouping Information | | | | | |
|---|---|---|---|---|---|
| Using the Tukey Method | | | | | |
| and 95% Confidence | | | | | |
| Group | N | Mean | Grouping | | |
| Blank MS | 3 | 5.0767 | A | | |
| IL-1B | 15 | 3.723 | A | | |
| SASC | 14 | 2.593 | | B | |
| ADSC | 9 | 2.055 | | B | C |
| Untreated | 15 | 1.0045 | | | C |
| Means that do not share | | | | | |
| a letter are significantly | | | | | |
| different. | | | | | |

Tukey Simultaneous Tests for Differences of Means

| | Difference | SE of | | | Adjusted |
|---|---|---|---|---|---|
| Difference of Levels | of Means | Difference | 95% CI | T-Value | P-Value |
| Blank MS − ADSC | 3.022 | 0.666 | (1.138, 4.906) | 4.54 | 0 |
| IL-1B − ADSC | 1.668 | 0.421 | (0.477, 2.860) | 3.96 | 0.002 |
| SASC − ADSC | 0.539 | 0.427 | (−0.668, 1.746) | 1.26 | 0.715 |
| Untreated − ADSC | −1.05 | 0.421 | (−2.242, 0.141) | −2.49 | 0.108 |
| IL-1B − Blank MS | −1.354 | 0.632 | (−3.141, 0.433) | −2.14 | 0.218 |
| SASC − Blank MS | −2.483 | 0.636 | (−4.281, −0.686) | −3.91 | 0.002 |
| Untreated − Blank MS | −4.072 | 0.632 | (−5.859, −2.285) | −6.45 | 0 |
| SASC − IL-1B | −1.13 | 0.371 | (−2.180, −0.080) | −3.04 | 0.029 |
| Untreated − IL-1B | −2.719 | 0.365 | (−3.750, −1.687) | −7.45 | 0 |
| Untreated − SASC | −1.589 | 0.371 | (−2.639, −0.539) | −4.28 | 0.001 |
| Individual confidence | | | | | |
| level = 99.33% | | | | | |
| Tukey Simultaneous | | | | | |
| 95% CIs | | | | | |

TABLE 5
PRG4 Fold Change ANOVA Summary

| Source | DF | Adj SS | Adj MS | F-Value | P-Value |
|---|---|---|---|---|---|
| Group | 4 | 571508 | 142877 | 101.63 | 0 |
| Error | 19 | 26710 | 1406 | | |
| Total | 23 | 598218 | | | |

Tukey Pairwise Comparisons

| Grouping Information | | | | | |
|---|---|---|---|---|---|
| Using the Tukey Method | | | | | |
| and 95% Confidence | | | | | |
| Group | N | Mean | Grouping | | |
| ADSC | 3 | 392.9 | A | | |
| IL-1B | 6 | 377.39 | A | | |
| Blank MS | 3 | 308.1 | A | | |
| SASC | 6 | 159.1 | | B | |
| Untreated | 6 | 1.0063 | | | C |
| Means that do not share | | | | | |
| a letter are significantly | | | | | |
| different. | | | | | |

Tukey Simultaneous Tests for Differences of Means

| | Difference | SE of | | | Adjusted |
|---|---|---|---|---|---|
| Difference of Levels | of Means | Difference | 95% CI | T-Value | P-Value |
| Blank MS − ADSC | −84.7 | 30.6 | (−176.7, 7.3) | −2.77 | 0.08 |
| IL-1B − ADSC | −15.5 | 26.5 | (−95.1, 64.2) | −0.58 | 0.976 |
| SASC − ADSC | −233.8 | 26.5 | (−313.5, −154.1) | −8.82 | 0 |
| Untreated − ADSC | −391.8 | 26.5 | (−471.5, −312.2) | −14.78 | 0 |
| IL-1B − Blank MS | 69.3 | 26.5 | (−10.4, 149.0) | 2.61 | 0.108 |
| SASC − Blank MS | −149.1 | 26.5 | (−228.7, −69.4) | −5.62 | 0 |
| Untreated − Blank MS | −307.1 | 26.5 | (−386.8, −227.4) | −11.58 | 0 |
| SASC − IL-1B | −218.3 | 21.6 | (−283.4, −153.3) | −10.09 | 0 |
| Untreated − IL-1B | −376.4 | 21.6 | (−441.4, −311.3) | −17.39 | 0 |
| Untreated − SASC | −158 | 21.6 | (−223.1, −93.0) | −7.3 | 0 |
| Individual confidence | | | | | |
| level = 99.27% | | | | | |
| Tukey Simultaneous | | | | | |
| 95% CIs | | | | | |

### Example 2

### Evaluating In Vivo Cartilage Regeneration Potential of SASC

The potential of SASC to attenuate enzyme induced articular cartilage degeneration was evaluated using an in vivo rodent model³¹ (FIG. 1B) with the contra-lateral knee taken as a healthy control. After OA induction, the animals were divided into 4 treatment groups (n=11): Osteoarthritis Control treated with Saline; Rats injected with 1 million ADSCs suspended in saline; Rats injected with 1 million blank particles in saline; Rats injected with 1 million SASC particles suspended in saline. The approximate dose of each growth factor in this group was 9.256 ng HGH and FGF-18, 11.86 ng IGF-1 and 5.424 ng TGF-β1.

### Joint Swelling

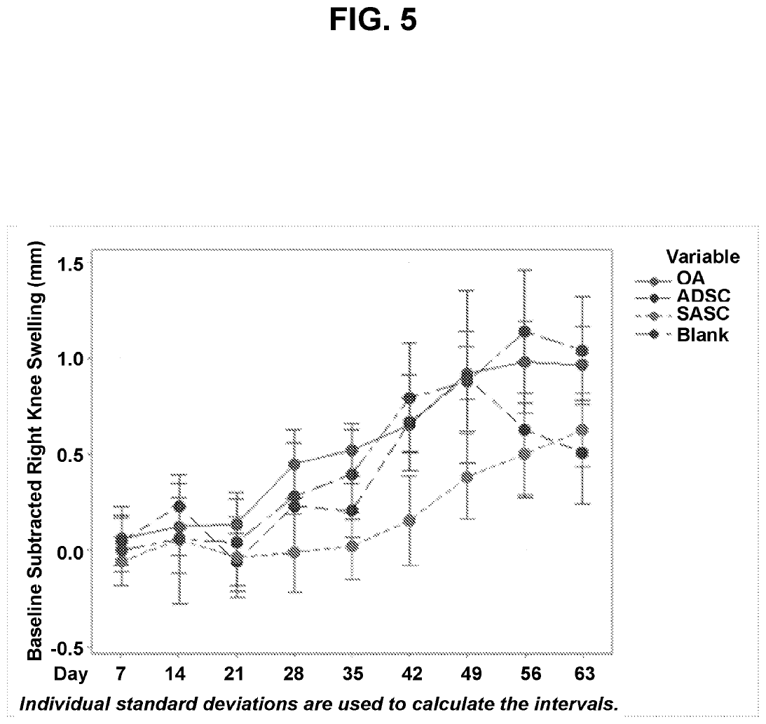

Progression of inflammation, as indicated by gross swelling (FIG. 5), was slowed upon the first treatment of ADSC and the synthetic artificial stem cells (SASC). Following the initial treatment injection, joint swelling for the OA control progressed significantly at day 28, one week before the second injection. While injection of the blank microspheres did have any effect on swelling, joint swelling in the ADSC and SASC injected joints were significantly lower than that of the OA group. Interestingly, one week after the second treatment injection (day 42), the extent of swelling increased in all groups (FIG. 5). While the SASC injected group continued to have lower swelling compared to the OA and blank groups, there was a continued progression of swelling between the second treatment injection and the end of the study. Two weeks post second injection, swelling in both ADSC and OA group were similar, however during the last two weeks of the study, the swelling in ADSC group significantly dropped to a level similar to SAS5 group.

### Investigating Effect on OA Attenuation by Safranin O Staining

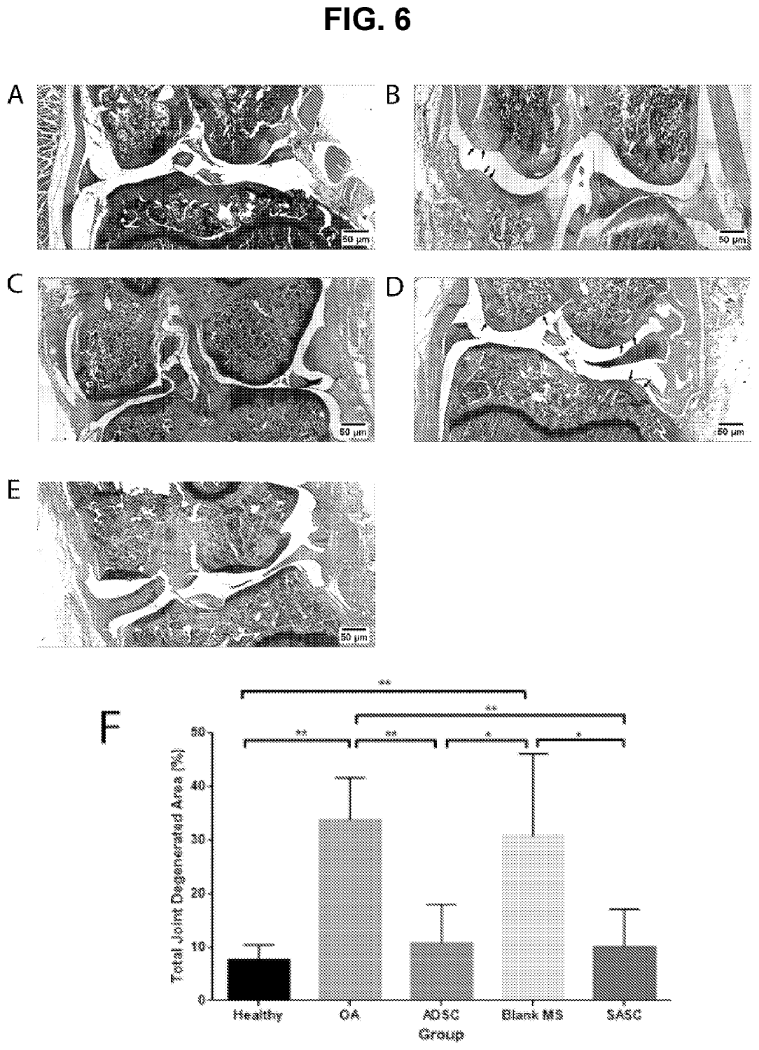

Frontal sections taken from the middle of the intact joint were stained with safranin O to assess the degenerated cartilage matrix area caused by the OA phenotype (FIG. 6). OA control animals showed loss of proteoglycan staining (FIG. 6B) as well as lesions and erosion at the articular surfaces (33.75±7.81% total degenerated area). SASC (FIG. 6E) and ADSC (FIG. 6C) injected groups (10.03±7.05% and 10.62±7.33% respectively) were comparable to the healthy joint (FIG. 6A) (7.53±2.84%), showing strong safranin O staining which indicated the SASC and ADSC treatments could significantly prevent loss of proteoglycan content of the cartilage ECM (FIG. 6F). Furthermore, treatment with the blank group (FIG. 6D) (30.61±15.45%) did not have any effect in recovering the matrix, showing more prominent lesions and areas of erosion with no signs of cartilage matrix preservation. This confirmed the preservative effect of SASC was due to the controlled release of the synthetic secretome.

TABLE 6
% Degenerated Area ANOVA Summary

| Source | DF | Adj SS | Adj MS | F-Value | P-Value |
|---|---|---|---|---|---|
| Group | 4 | 3169 | 792.17 | 9.63 | 0 |
| Error | 20 | 1645 | 82.24 | | |
| Total | 24 | 4814 | | | |

Tukey Pairwise Comparisons

| Grouping Information | | | | |
|---|---|---|---|---|
| Using the Tukey Method | | | | |
| and 95% Confidence | | | | |
| Group | N | Mean | Grouping | |
| OA | 5 | 33.75 | A | |
| Blank | 5 | 30.61 | A | |
| ADSC | 5 | 10.62 | | B |
| SASC | 5 | 10.03 | | B |
| Healthy | 5 | 7.53 | | B |
| Means that do not share a | | | | |
| letter are significantly | | | | |
| different. | | | | |

Tukey Simultaneous Tests for Differences of Means

| | Difference | SE of | | | Adjusted |
|---|---|---|---|---|---|
| Difference of Levels | of Means | Difference | 95% CI | T-Value | P-Value |
| Blank − ADSC | 19.99 | 5.74 | (2.84, 37.15) | 3.49 | 0.018 |
| Healthy − ADSC | −3.09 | 5.74 | (−20.25, 14.06) | −0.54 | 0.982 |
| OA − ADSC | 23.13 | 5.74 | (5.98, 40.29) | 4.03 | 0.005 |
| SASC − ADSC | −0.6 | 5.74 | (−17.75, 16.56) | −0.1 | 1 |
| Healthy − Blank | −23.09 | 5.74 | (−40.24, −5.93) | −4.03 | 0.005 |
| OA − Blank | 3.14 | 5.74 | (−14.02, 20.29) | 0.55 | 0.981 |
| SASC − Blank | −20.59 | 5.74 | (−37.74, −3.43) | −3.59 | 0.014 |
| OA − Healthy | 26.23 | 5.74 | (9.07, 43.38) | 4.57 | 0.002 |
| SASC − Healthy | 2.5 | 5.74 | (−14.66, 19.65) | 0.44 | 0.992 |
| SASC − OA | −23.73 | 5.74 | (−40.88, −6.57) | −4.14 | 0.004 |
| Individual confidence | | | | | |
| level = 99.28% | | | | | |
| Tukey Simultaneous | | | | | |
| 95% CIs | | | | | |

### Evaluating Biomechanical Outcomes by Nano-Indentation

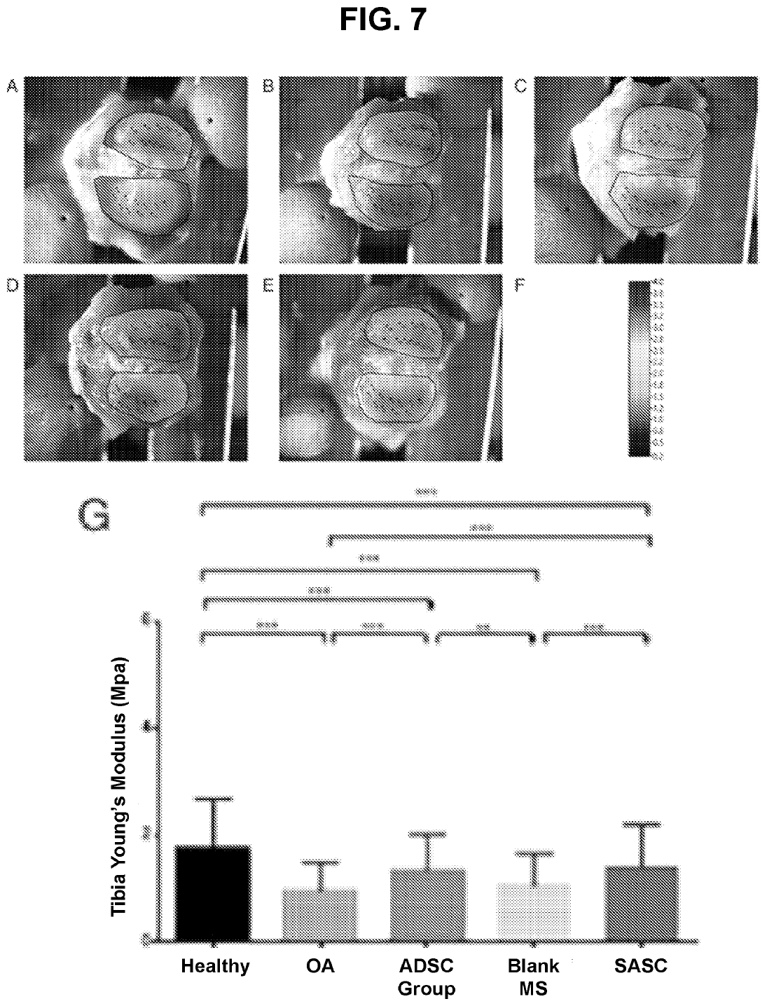

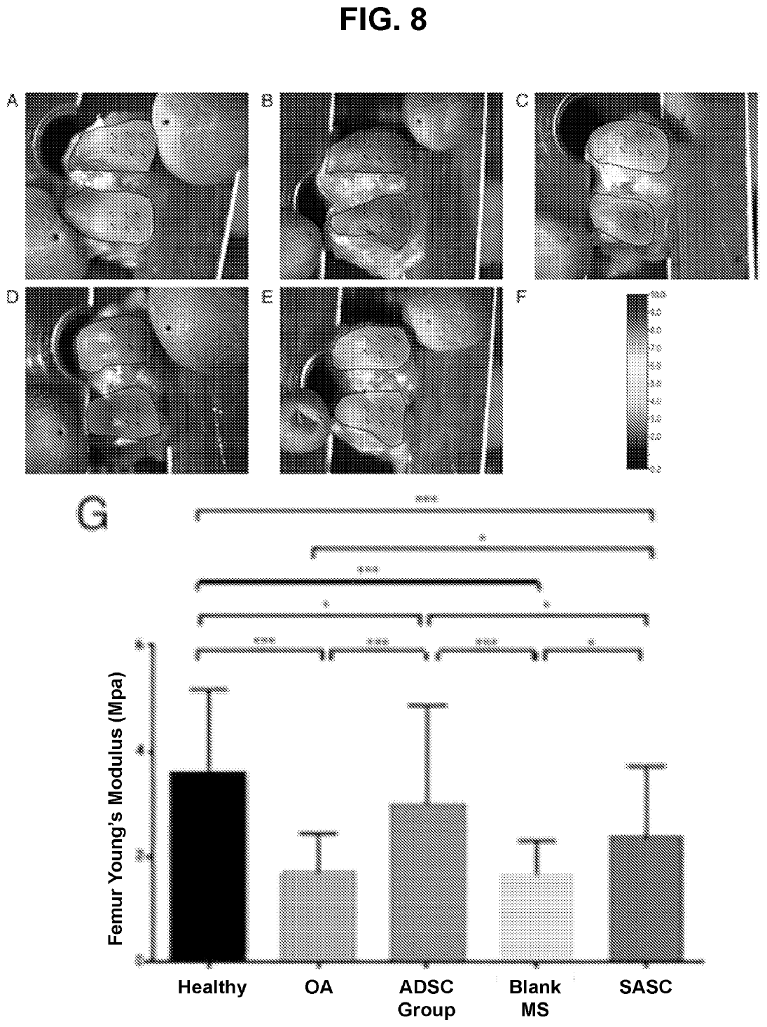

Heat maps produced for the average stiffness at each surface (FIGS. 7, 8) and statistical differences of the surface stiffness between groups were identified (FIG. 7G, 8G). OA cartilage had a significantly lower modulus (Tibia: 0.96±0.53 MPa; Femur: 1.70±0.74 Mpa) compared to the contralateral joint (Tibia: 1.79±0.61 Mpa; Femur: 3.61±1.56 Mpa) taken as a healthy control (FIG. 7A-B, 8A-B). Treatment with ADSC (Tibia: 1.36±0.66 Mpa; Femur: 3.00±1.88 Mpa) and SASC (FIG. 7C&E, 8 C&E) (Tibia: 1.41±0.79 Mpa; Femur: 2.35±1.37 Mpa) resulted in an increased modulus, indicating recovery of cartilage stiffness though this recovery was not similar to the healthy control using either treatment. Lastly, the blank group (FIG. 7D; 8D) (Tibia: 1.05±0.61 Mpa; Femur: 1.68±0.62 Mpa) did not have any significant impact on the young's modulus on either surface.

TABLE 7
Tibial Young's Modulus Linear Mixed Effects Model Summary

| Term | DF Num | DF Den | F-Value | P-Value |
|---|---|---|---|---|
| Group | 4 | 627.05 | 35.81 | 0 |

Tukey Pairwise Comparisons: Group

| Grouping Information | | | | | |
|---|---|---|---|---|---|
| Using the Tukey | | | | | |
| Method and 95% | | | | | |
| Confidence | | | | | |
| Group | N | Mean | Grouping | | |
| Healthy | 131 | 1.79277 | A | | |
| SASC | 132 | 1.41259 | | B | |
| ADSC | 132 | 1.35554 | | B | |
| Blank | 132 | 1.05128 | | | C |
| OA | 131 | 0.96371 | | | C |
| Means that do not | | | | | |
| share a letter | | | | | |
| are significantly | | | | | |
| different. | | | | | |

| Difference of Group | Difference | SE of | | Simultaneous | T- | Adjusted |
|---|---|---|---|---|---|---|
| Levels | of Means | Difference | DF | 95% CI | Value | P-Value |
| Blank - ADSC | −0.3043 | 0.0774 | 627.022 | (−0.5161, −0.0924) | −3.93 | 0.001 |
| Healthy - ADSC | 0.4372 | 0.0776 | 627.059 | (0.2249, 0.6495) | 5.63 | 0 |
| OA - ADSC | −0.3918 | 0.0776 | 627.059 | (−0.6041, −0.1795) | −5.05 | 0 |
| SASC - ADSC | 0.0571 | 0.0774 | 627.022 | (−0.1548, 0.2689) | 0.74 | 0.948 |
| Healthy - Blank | 0.7415 | 0.0776 | 627.059 | (0.5292, 0.9538) | 9.56 | 0 |
| OA - Blank | −0.0876 | 0.0776 | 627.059 | (−0.2999, 0.1247) | −1.13 | 0.791 |
| SASC - Blank | 0.3613 | 0.0774 | 627.022 | (0.1494, 0.5732) | 4.67 | 0 |
| OA - Healthy | −0.8291 | 0.0778 | 627.105 | (−1.0418, −0.6163) | −10.66 | 0 |
| | | | | | 10.66 | |
| SASC - Healthy | −0.3802 | 0.0776 | 627.059 | (−0.5925, −0.1679) | −4.9 | 0 |
| SASC - OA | 0.4489 | 0.0776 | 627.059 | (0.2366, 0.6612) | 5.78 | 0 |
| Individual confidence | | | | | | |
| level = 99.36% | | | | | | |
| Tukey Simultaneous | | | | | | |
| 95% CIs | | | | | | |

TABLE 8
Femoral Young's Modulus Linear Mixed Effects Model Summary

| Term | DF Num | DF Den | F-Value | P-Value |
|---|---|---|---|---|
| Group | 4 | 320.09 | 28.86 | 0 |

Tukey Pairwise Comparisons: Group

| Grouping | | | | | | |
|---|---|---|---|---|---|---|
| Information Using | | | | | | |
| the Tukey Method | | | | | | |
| and 95% Confidence | | | | | | |
| Group | N | Mean | Grouping | | | |
| Healthy | 72 | 3.61372 | A | | | |
| ADSC | 68 | 3.00305 | | B | | |
| SASC | 70 | 2.34487 | | | C | |
| OA | 65 | 1.69795 | | | | D |
| Blank | 65 | 1.69002 | | | | D |
| Means that do not | | | | | | |
| share a letter are | | | | | | |
| significantly | | | | | | |
| different. | | | | | | |

| Difference of | Difference | SE of | | Simultaneous | | Adjusted |
|---|---|---|---|---|---|---|
| Group Levels | of Means | Difference | DF | 95% CI | T-Value | P-Value |
| Blank - ADSC | −1.313 | 0.224 | 320.763 | (−1.928, −0.698) | −5.85 | 0 |
| Healthy - ADSC | 0.611 | 0.218 | 319.596 | (0.012, 1.210) | 2.8 | 0.043 |
| OA - ADSC | −1.305 | 0.224 | 320.812 | (−1.921, −0.690) | −5.82 | 0 |
| SASC - ADSC | −0.658 | 0.22 | 319.848 | (−1.262, −0.055) | −2.99 | 0.025 |
| Healthy - Blank | 1.924 | 0.221 | 320.143 | (1.317, 2.530) | 8.7 | 0 |
| OA - Blank | 0.008 | 0.227 | 320.66 | (−0.614, 0.630) | 0.03 | 1 |
| SASC - Blank | 0.655 | 0.223 | 320.552 | (0.044, 1.266) | 2.94 | 0.029 |
| OA - Healthy | −1.916 | 0.221 | 319.751 | (−2.522, −1.310) | −8.67 | 0 |
| SASC - Healthy | −1.269 | 0.217 | 319.013 | (−1.863, −0.675) | −5.86 | 0 |
| SASC - OA | 0.647 | 0.222 | 319.855 | (0.037, 1.257) | 2.91 | 0.032 |
| Individual | | | | | | |
| confidence level = | | | | | | |
| 99.36% | | | | | | |
| Tukey | | | | | | |
| Simultaneous 95% | | | | | | |
| CIs | | | | | | |

### Discussion

Stem cells have been widely used in regenerative engineering due to their multipotent ability as well as their capability to produce paracrine factors that affect the local microenvironment. However, sensitivity to changes in the local microenvironment may lead to spontaneous changes in cellular function and the secreted factor composition, leading to challenges in translatability. In this study, a completely synthetic, tailored alternative to the stem cell was engineered by loading certain factors found in the stem cell secretome into a PLGA matrix. As a pilot system, the SASC system was formulated and evaluated to attenuate Osteoarthritis. Herein, IGF-1, TGF-β1, HGH and FGF-18 were chosen for developing the SASC system.

All of these growth factors were loaded using a standardized double emulsion (W/O/W) method that surprisingly resulted in similar microsphere yields in the average size of a mesenchymal stem cell (about 10-20 μm) 38 (FIG. 1) (about 70% of the resulting microspheres had a diameter of about 10-20 μm). Having a standardized fabrication method in which yield is not dependent on the factor being loaded proves greatly advantageous when looking at the versatility of SASC in which different growth factors may be used for different applications. Also disclosed herein is a successful demonstration of a standardized process to load different factors while maintaining microsphere size minimizes optimization needed to load individual growth factors into microspheres.

As shown herein, IGF-1 (about 7 kDa) had a high loading efficiency into these microspheres without the need for a carrier protein, and also released almost completely from the microspheres within 28 days. The higher molecular weight and larger HGH and TGF-β1 molecules, having about 20 and 40 kDa respectively, required the use of a carrier protein (BSA) to be loaded into the microspheres and while 60% of each factor released from the PLGA microspheres, the profile of TGF-β1 was much more linear compared to the burst and subsequent plateau of HGH release, indicating that the larger size and slightly more hydrophobic nature of TGF-β1 may have played a role in hindering diffusion out of the microsphere. While all of these factors had different loading efficiencies, SEM images (FIG. 2), confirmed that microsphere physical characteristics were not affected and therefore loading efficiency could be a function of the biophysical properties of the factor being loaded. Because FGF-18 (about 20 kDa) has similar size to HGH it should have similar loading properties and used the same amount of carrier protein.

In this study, osteoarthritis was used as a target model and a synthetic artificial stem cell (SASC) system was engineered consisting of growth factors that are able to stimulate ECM synthesis as well as reduce inflammation to disrupt the cycle of inflammation/degeneration. In the osteoarthritic joint, the homeostasis has been shifted towards catabolic processes, with the presence of M1 macrophages in the synovium stimulated by pro-inflammatory cytokines such as IL-β0 and Tumor necrosis factor-alpha (TNF-α)^{39,40}. This stimulation perpetuates inflammation of the synovial membrane through the synthesis of inflammatory mediators such as matrix degrading enzymes like ADAMTS5 and various matrix metalloproteases (MMPs). These enzymes then diffuse through the synovial fluid into the cartilage and start a vicious cycle of inflammation and degeneration characterized by chondrocyte hypertrophy and hypocellularity^{40,41}.

In vitro characterization has shown that SASC is able to inhibit catabolic processes as a result of inflammation in chondrocytes. In this in vitro study, inflammation was induced in rat chondrocytes with IL-1β which bears a close resemblance to the inflammatory process of the cartilage during osteoarthritis⁴². The paracrine effects of ADSC to those of SASC were compared in a co-culture transwell system (FIG. 1A). Nitric oxide (NO) is an inflammatory product generated through the inducible NOS (iNOS) pathway, which inhibits the synthesis of collagen and proteoglycans and increases activity of proteolytic enzymes. NO production in the IL-1β group increased with 20 ng/mL of IL-1β. Treatment with SASC particles for 3 days significantly decreased NO production (FIG. 4A) which can potentially be attributed to the suppression of the iNOS pathway with released TGF-β1⁴³. Generally arthritic chondrocytes are less responsive to IGF-1 because of the presence of iNOS⁴⁴. It's possible in this system that TGF-β1 reduced the expression of iNOS which further enhanced the chondrocyte response to anabolic IGF-1, resulting in suppression of downstream inflammatory pathways. This led to a more potent anti-inflammatory effect of SASC compared to the paracrine factors produced by ADSCs which may also include a wide variety of iNOS inducing factors (such as vascular endothelial growth factor).

As an effect of IL-1β inflammation induction, expression of the master chondrogenic transcription factor, SOX9 was downregulated and aggrecanase ADAMTS5 was increased. SOX9 signals for early chondrogenesis, leading to the production of ECM proteoglycans such as Collagen II and aggrecan. The downregulation of SOX9 by IL-1β indicates a loss of early chondrocytes and indicates hypocellularity could occur. SASC's potent upregulation of SOX9 (FIG. 4B) may be attributed to the presence of high IGF-1 and FGF-18, factors important to chondrocyte proliferation and ECM production^{24-26,33,44}. Lubricin or PRG4 serves as both a boundary lubricant and a chondroprotective agent⁴⁵. Previous reports have shown decreased lubricin concentration in ACL transection induced osteoarthritis in rodents and humans as well as in humans with late-stage chronic OA. On the contrary, there are studies conducted in sheep, dogs and horses that have observed an increase of PRG4 in OA models emphasizing the complex and sensitive autocrine role PRG4 plays in modulating progression of osteoarthritis⁴⁵⁻⁵⁰. An increase in PRG4 upon IL-1β induced inflammation was observed and may indicate a negative feedback regulation of inflammation. Interestingly, in addition to having a similar anti-inflammatory effect compared to ADSCs (reducing inflammatory effects ADAMTS5^{51,52}), SASC was able to reverse the effects of inflammation on NO, SOX9 and PRG4 (FIG. 4A-D). Therefore, SASC is shown to contribute a more potent chondro-protective effect as a result of the targeted composition of growth factors used in this composition.

A collagenase induced OA model was used to evaluate the in vivo chondroprotective potential of SASC. This model has been established to investigate the mechanisms of OA pathogenesis31. Collagenase digests the collagen directly from cartilage ECM resulting in similar osteoarthritic changes such as pain, changes in synovial membrane, subchondral bone remodelling and degeneration of articular cartilage.

Blank microspheres were added in this composition to simulate the possibility of adding or removing factors that might be necessary for better tailor therapeutic outcomes for different tissues. In this case, it was determined that equal weight fractions of growth factor loaded spheres (20% w/w for each) could be combined to deliver the calculated doses in vivo.

Previous studies have demonstrated the advantages and positive outcomes of combining one or two factors^{35,60} in chondrogenesis, though to date, there have been no studies conducted to develop a mimic for the paracrine effect of a stem cell. Additionally, by harnessing different factor release patterns and loading efficiencies, SASC can be formulated using different weight ratios and tailored to become a unique synthetic cell therapy for other degenerative diseases or regenerative applications.

Joint swelling is one of the most prominent symptoms of osteoarthritis. It is usually indicative of synovitis, as the synovium within the joint may accumulate a number of inflammatory mediators which may contribute to the pain and further cartilage degeneration during OA⁶¹.

Although the inflammatory response to blank particles was similar to the OA group as seen in FIGS. 5 and 6, it's possible that this response is mediated by collagenase and not the PLGA matrix. The clinical use of PLGA as a biocompatible vehicle for sustained release of therapeutics has been thoroughly investigated in prior studies. Zhang et al. observed injection of PLGA microspheres induced a mild inflammatory response one day post injection which subsided after one week⁶². Other clinical trials have also indicated that PLGA microspheres do not induce any deleterious effects on cartilage or any aspects of joint structure over a period of 24 weeks⁶³. Therefore it's not likely that PLGA blank microspheres would evoke any additional inflammatory response. In addition, studies have shown the inflammatory response is size-dependent to micro/nano particles, demonstrating that smaller molecules (<5 μm diameter) are extensively phagocytosed by macrophages in the epithelial synovial lining which induce a higher inflammatory response in the synovial tissue⁶⁴⁻⁶⁶. As microspheres used herein were in the 10-20 μm range (similar to the size of mesenchymal stem cells), we do not anticipate phagocytosis-mediated inflammation.

On 28 days after the first injection, ADSCs and SASC resulted in a significant decrease compared to OA and blank controls. A more pronounced swelling was observed two weeks after the second treatment (at day 49) in both the treatment groups. Though the swelling in the ADSC group decreased after 49 days, swelling in the SASC group continued to increase incrementally (FIG. 5). This difference between ADSC and SASC was not significant and both remained significantly lower compared to the OA group by day 63. This response in the SASC group may be due to increased macrophage activity in response to increased polymer load in the joint but whether these macrophages are pro-inflammatory M1 macrophages or anti-inflammatory M2 macrophages⁶⁷ is yet to be determined. However, the decrease in cartilage degeneration (FIG. 6F) could be attributed to a shift towards higher M2 activity in the swollen joints.

SASC was also shown to attenuate degenerative OA to a similar extent as ADSCs in vivo. Safranin O staining used to visualize degenerated articular cartilage matrix showed little to no degeneration in harvested SASC and ADSC samples, similar to the contralateral joints as healthy control. Nano-indentation on the surface of the cartilage was done to investigate whether the resulting cartilage was biomechanically superior to OA cartilage and similar to healthy articular cartilage. As a result of the degenerated cartilage solid network in OA, the stiffness of the cartilage (as measured by the young's modulus) is decreased^{68,69}. While PLGA microspheres with PBS did not recover the young's modulus, ADSCs and SASC significantly increased the tibial and femoral modulus compared to OA (FIG. 7, 8). However, neither modulus was similar to that of the healthy contralateral tibia nor femur, indicating potential fibrillation of the cartilage that could be addressed in future SASC compositions.

In conclusion, the novel synthetic artificial stem cell (SASC) system disclosed herein opens possibilities to tailor paracrine responses of different cells as well as provide a more potent regenerative effect for targeted tissues. Importantly, due to its small size, SASC is a minimally invasive, injectable therapy that can be injected in high particle concentrations as well as the ability to combine multiple growth factor loaded microspheres in different weight ratios to tailor the composition in smaller injection volumes. Additionally, the synthetic nature of SASC mitigates risk associated with immunogenicity as well as variability in stem cell paracrine response due to donor properties. SASC is the first synthetic system to use a tailored cocktail of biological factors designed to mimic the paracrine effect of a stem cell. These studies also suggest that SASC may be a clinically translatable stem cell substitute. In vitro, SASC exhibits similar anti-inflammatory and chondro-protective effects as ADSCs on IL-1β inflamed chondrocytes. In vivo, two treatments of SASC have successfully attenuated cartilage degeneration resulting from enzyme-induced osteoarthritis and this cartilage is mechanically superior to OA cartilage though still less stiff than healthy cartilage. This suggests that SASC can provide a comparable effect to the ADSCs. To better understand how SASC affects the local joint environment, a mechanistic investigation should be done along with identification of any potential systemic anti-inflammatory responses and tracking the fate of SASC after injection.

The introduction of SASC shifts the paradigm of stem cell therapy into the synthetic domain. Loading of a recombinant secretome into a synthetic matrix provides a tailorable technology for many different areas of regenerative engineering. While this disclosure focuses on the application of SASC in attenuating OA progression, other systemic degenerative diseases may be evaluated. Furthermore, regeneration of various tissues requires different types of scaffolds, many of which stem cells are seeded to provide a biological cue to initiate regeneration. Combining SASC into such scaffolds would enhance retention in a local environment and provide these required cues exactly as seeded cells.

### REFERENCES

1. Vernekar, V. N., James, R., Smith, K. J. & Laurencin, C. T. Nanotechnology applications in stem cell science for regenerative engineering. Journal of Nanoscience and Nanotechnology vol. 16 8953-8965 (2016).
2. Laurencin, Cato T., Khan, Y. Regenerative Engineering—Google Books. (CRC Press/Taylor & Francis Group, 2013).
3. Laurencin, C. T. & Khan, Y. Regenerative engineering. Science Translational Medicine vol. 4 (2012).
4. Caplan, A. I. Review: Mesenchymal Stem Cells: Cell-Based Reconstructive Therapy in Orthopedics. Tissue Engineering 11, 1198-1211 (2005).
5. Shah, S., Otsuka, T., Bhattacharjee, M. & Laurencin, C. T. Minimally Invasive Cellular Therapies for Osteoarthritis Treatment. Regen. Eng. Transl. Med. (2020) doi:10.1007/s40883-020-00184-w.
6. Naji, A. et al. Biological functions of mesenchymal stem cells and clinical implications. Cellular and Molecular Life Sciences vol. 76 3323-3348 (2019).
7. Krueger, T. E. G., Thorek, D. L. J., Denmeade, S. R., Isaacs, J. T. & Brennen, W. N. Concise Review: Mesenchymal Stem Cell-Based Drug Delivery: The Good, the Bad, the Ugly, and the Promise. Stem Cells Translational Medicine vol. 7 651-663 (2018).
8. Narayanan, G., Bhattacharjee, M., Nair, L. S. & Laurencin, C. T. Musculoskeletal Tissue Regeneration: the Role of the Stem Cells. Regenerative Engineering and Translational Medicine vol. 3 133-165 (2017).
9. Turinetto, V., Vitale, E. & Giachino, C. Senescence in Human Mesenchymal Stem Cells: Functional Changes and Implications in Stem Cell-Based Therapy. International Journal of Molecular Sciences 17, 1164 (2016).
10. Lukomska, B. et al. Challenges and Controversies in Human Mesenchymal Stem Cell Therapy. Stem Cells International vol. 2019 (2019).
11. Laurencin, C. T. & McClinton, A. Regenerative Cell-Based Therapies: Cutting Edge, Bleeding Edge, and Off the Edge. Regenerative Engineering and Translational Medicine 6, 78-89 (2020).
12. Mitchell, R. et al. Secretome of adipose-derived mesenchymal stem cells promotes skeletal muscle regeneration through synergistic action of extracellular vesicle cargo and soluble proteins. Stem Cell Res Ther 10, 116 (2019).
13. Maguire, G. Stem cell therapy without the cells. Communicative & Integrative Biology 6, e26631 (2013).
14. Zhou, Y., Yamamoto, Y., Xiao, Z. & Ochiya, T. The Immunomodulatory Functions of Mesenchymal Stromal/Stem Cells Mediated via Paracrine Activity. Journal of Clinical Medicine 8, 1025 (2019).
15. Menasche, P. Cell therapy trials for heart regeneration—lessons learned and future directions. Nature Reviews Cardiology vol. 15 659-671 (2018).
16. Eggenhofer, E., Luk, F., Dahlke, M. H. & Hoogduijn, M. J. The Life and Fate of Mesenchymal Stem Cells. Frontiers in Immunology 5, 148 (2014).
17. Daneshmandi, L. et al. Emergence of the Stem Cell Secretome in Regenerative Engineering. Trends in Biotechnology S0167779920301189 (2020) doi :10.1016/j .tibtech.2020.04.013.
18. Eleuteri, S. & Fierabracci, A. Insights into the Secretome of Mesenchymal Stem Cells and Its Potential Applications. IDMS 20, 4597 (2019).
19. Vizoso, F., Eiro, N., Cid, S., Schneider, J. & Perez-Fernandez, R. Mesenchymal Stem Cell Secretome: Toward Cell-Free Therapeutic Strategies in Regenerative Medicine. International Journal of Molecular Sciences 18, 1852 (2017).
20. Van Pham, P., Nguyen, H. T. & Vu, N. B. Evolution of Stem Cell Products in Medicine: Future of Off-the-Shelf Products. in 93-118 (Springer, Cham, 2018). doi:10.1007/978-3-319-99328-76.
21. Katagiri, W., Osugi, M., Kawai, T. & Hibi, H. First-in-human study and clinical case reports of the alveolar bone regeneration with the secretome from human mesenchymal stem cells. Head Face Med 12, 5 (2016).
22. Katagiri, W., Osugi, M., Kinoshita, K. & Hibi, H. Conditioned Medium From Mesenchymal Stem Cells Enhances Early Bone Regeneration After Maxillary Sinus Floor Elevation in Rabbits: Implant Dentistry 1 (2015) doi:10.1097/ID.0000000000000335.
23. Katagiri, W. et al. A defined mix of cytokines mimics conditioned medium from cultures of bone marrow-derived mesenchymal stem cells and elicits bone regeneration. Cell Prolif 50, e12333 (2017).
24. Davidson, D. et al. Fibroblast Growth Factor (FGF) 18 Signals through FGF Receptor 3 to Promote Chondrogenesis. Journal of Biological Chemistry 280, 20509-20515 (2005).
25. Ellman, M. B. et al. Fibroblast growth factor control of cartilage homeostasis. J. Cell. Biochem. 114, 735-742 (2013).
26. Moore, E. E. et al. Fibroblast growth factor-18 stimulates chondrogenesis and cartilage repair in a rat model of injury-induced osteoarthritis. Osteoarthritis and Cartilage 13, 623-631 (2005).
27. Bergan-Roller, H. E. & Sheridan, M. A. The growth hormone signaling system: Insights into coordinating the anabolic and catabolic actions of growth hormone. General and Comparative Endocrinology 258, 119-133 (2018).
28. Carter-Su, C., Schwartz, J. & Argetsinger, L. S. Growth hormone signaling pathways. Growth Hormone & IGF Research 28, 11-15 (2016).
29. Rui, J. et al. Controlled release of vascular endothelial growth factor using poly-lactic-co-glycolic acid microspheres: In vitro characterization and application in polycaprolactone fumarate nerve conduits. Acta Biomaterialia 8, 511-518 (2012).
30. Dinarvand, R., Moghadam, S. H., Sheikhi, A. & Atyabi, F. Effect of surfactant HLB and different formulation variables on the properties of poly-D,L-lactide microspheres of naltrexone prepared by double emulsion technique. Journal of Microencapsulation 22, 139-151 (2005).
31. Adães, S. et al. Intra-articular injection of collagenase in the knee of rats as an alternative model to study nociception associated with osteoarthritis. Arthritis Res Ther 16, R10 (2014).
32. O'Connor, W. J., Botti, T., Khan, S. N. & Lane, J. M. THE USE OF GROWTH FACTORS IN CARTILAGE REPAIR. Orthopedic Clinics of North America 31, 399-409 (2000).
33. Shakibaei, M., Seifarth, C., John, T., Rahmanzadeh, M. & Mobasheri, A. Igf-I extends the chondrogenic potential of human articular chondrocytes in vitro: Molecular association between Sox9 and Erk1/2. Biochemical Pharmacology 72, 1382-1395 (2006).
34. W, W., D, R. & Lyons, K. TGFB Signaling in Cartilage Development and Maintenance. Birth Defects Res C Embryo Today 102, 37-51 (2014).
35. Jaklenec, A. et al. Sequential release of bioactive IGF-I and TGF-β1 from PLGA microsphere-based scaffolds. Biomaterials 29, 1518-1525 (2008).
36. Carter-Su, C., Schwartz, J. & Argetsinger, L. S. Growth hormone signaling pathways. Growth Hormone and IGF Research 28, 11-15 (2016).
37. Bergan-Roller, H. E. & Sheridan, M. A. The growth hormone signaling system: Insights into coordinating the anabolic and catabolic actions of growth hormone. General and Comparative Endocrinology 258, 119-133 (2018).
38. Bora, P. & Majumdar, A. S. Adipose tissue-derived stromal vascular fraction in regenerative medicine: a brief review on biology and translation. Stem Cell Res Ther 8, 145 (2017).
39. Chen, Y. et al. Macrophages in osteoarthritis: pathophysiology and therapeutics. Am J Transl Res 12, 261-268 (2020).
40. Zhang, H., Cai, D. & Bai, X. Macrophages regulate the progression of osteoarthritis. Osteoarthritis and Cartilage 28, 555-561 (2020).
41. Liu, B., Zhang, M., Zhao, J., Zheng, M. & Yang, H. Imbalance of M1/M2 macrophages is linked to severity level of knee osteoarthritis. Exp Ther Med (2018) doi:10.3892/etm.2018.6852.
42. Daheshia, M. & Yao, J. Q. The interleukin 1β pathway in the pathogenesis of osteoarthritis. Journal of Rheumatology vol. 35 2306-2312 (2008).
43. Takaki, H. et al. TGF-β1 suppresses IFN-γ-induced NO production in macrophages by suppressing STAT1 activation and accelerating iNOS protein degradation. Genes Cells 11, 871-882 (2006).
44. Studer, R. K. Nitric oxide decreases IGF-1 receptor function in vitro; glutathione depletion enhances this effect in vivo. Osteoarthritis and Cartilage 12, 863-869 (2004).
45. Reesink, H. L., Watts, A. E., Mohammed, H. O., Jay, G. D. & Nixon, A. J. Lubricin/proteoglycan 4 increases in both experimental and naturally occurring equine osteoarthritis. Osteoarthritis and Cartilage 25, 128-137 (2017).
46. Desrochers, J., Amrein, M. W. & Matyas, J. R. Microscale surface friction of articular cartilage in early osteoarthritis. Journal of the Mechanical Behavior of Biomedical Materials 25, 11-22 (2013).
47. Young, A. A. et al. Proteoglycan 4 downregulation in a sheep meniscectomy model of early osteoarthritis. Arthritis Research and Therapy 8, 4-9 (2006).
48. Teeple, E. et al. Coefficients of friction, lubricin, and cartilage damage in the anterior cruciate ligament-deficient guinea pig knee. Journal of Orthopaedic Research 26, 231-237 (2008).
49. Elsaid, K. A. et al. Decreased lubricin concentrations and markers of joint inflammation in the synovial fluid of patients with anterior cruciate ligament injury. Arthritis and Rheumatism 58, 1707-1715 (2008).
50. Elsaid, K. A., Machan, J. T., Waller, K., Fleming, B. C. & Jay, G. D. The impact of anterior cruciate ligament injury on lubricin metabolism and the effect of inhibiting tumor necrosis factor α on chondroprotection in an animal model. Arthritis and Rheumatism 60, 2997-3006 (2009).
51. Bhattacharjee, M. et al. Preparation and characterization of amnion hydrogel and its synergistic effect with adipose derived stem cells towards IL1β activated chondrocytes. Sci Rep 10, 18751 (2020).
52. Sun, Z., Nair, L. S. & Laurencin, C. T. The Paracrine Effect of Adipose-Derived Stem Cells Inhibits IL-1β-induced Inflammation in Chondrogenic Cells through the Wnt/β-Catenin Signaling Pathway. Regen. Eng. Transl. Med. 4, 35-41 (2018).
53. Schmidt, M. B., Chen, E. H. & Lynch, S. E. A review of the effects of insulin-like growth factor and platelet derived growth factor on in vivo cartilage healing and repair. Osteoarthritis and Cartilage 14, 403-412 (2006).
54. Loffredo, F. S. et al. Targeted Delivery to Cartilage Is Critical for In Vivo Efficacy of Insulin-like Growth Factor 1 in a Rat Model of Osteoarthritis: Efficacy of Cartilage-Targeted Delivery. Arthritis & Rheumatology 66, 1247-1255 (2014).
55. Zhang, Z. et al. The effects of different doses of IGF-1 on cartilage and subchondral bone during the repair of full-thickness articular cartilage defects in rabbits. Osteoarthritis and Cartilage 25, 309-320 (2017).
56. Danna, N. R. et al. The Effect of Growth Hormone on Chondral Defect Repair. CARTILAGE 9, 63-70 (2018).
57. Kwon, D. R. & Park, G. Y. Effect of Intra-articular Injection of AOD9604 with or without Hyaluronic Acid in Rabbit Osteoarthritis Model. 45, 7 (2015).
58. Reker, D. et al. Sprifermin (rhFGF18) versus vehicle induces a biphasic process of extracellular matrix remodeling in human knee OA articular cartilage ex vivo. Sci Rep 10, 6011 (2020).
59. Moore, E. E. et al. Fibroblast growth factor-18 stimulates chondrogenesis and cartilage repair in a rat model of injury-induced osteoarthritis. Osteoarthritis and Cartilage 13, 623-631 (2005).
60. Solorio, L. D., Vieregge, E. L., Dhami, C. D. & Alsberg, E. High-Density Cell Systems Incorporating Polymer Microspheres as Microenvironmental Regulators in Engineered Cartilage Tissues. Tissue Engineering Part B: Reviews 19, 209-220 (2013).
61. Mora, J. C., Przkora, R. & Cruz-Almeida, Y. Knee osteoarthritis: pathophysiology and current treatment modalities. JPR Volume 11, 2189-2196 (2018).
62. Zhang, Z. & Huang, G. Intra-articular lornoxicam loaded PLGA microspheres: enhanced therapeutic efficiency and decreased systemic toxicity in the treatment of osteoarthritis. Drug Delivery 19, 255-263 (2012).
63. Paik, J., Duggan, S. T. & Keam, S. J. Triamcinolone Acetonide Extended-Release: A Review in Osteoarthritis Pain of the Knee. Drugs 79, 455-462 (2019).
64. Pradal, J. et al. Effect of particle size on the biodistribution of nano- and microparticles following intra-articular injection in mice. International Journal of Pharmaceutics 498, 119-129 (2016).
65. Horisawa, E. et al. Prolonged Anti-Inflammatory Action of DL-Lactide/Glycolide Copolymer Nanospheres Containing Betamethasone Sodium Phosphate for an Intra-Articular Delivery System in Antigen-Induced Arthritic Rabbit. Pharmaceutical Research 19, 403-410 (2002).
66. Horisawa, E. et al. Size-Dependency of DL-Lactide/Glycolide Copolymer Particulates for Intra-Articular Delivery System on Phagocytosis in Rat Synovium. Pharmaceutical Research 19, 132-139 (2002).
67. Liu, B., Zhang, M., Zhao, J., Zheng, M. & Yang, H. Imbalance of M1/M2 macrophages is linked to severity level of knee osteoarthritis. Exp Ther Med (2018) doi:10.3892/etm.2018.6852.
68. Doyran, B. et al. Nanoindentation modulus of murine cartilage: a sensitive indicator of the initiation and progression of post-traumatic osteoarthritis. Osteoarthritis and Cartilage 25, 108-117 (2017).
69. Peters, A. E., Akhtar, R., Comerford, E. J. & Bates, K. T. The effect of ageing and osteoarthritis on the mechanical properties of cartilage and bone in the human knee joint. Sci Rep 8, 5931 (2018).
Katagiri W, Sakaguchi K, Kawai T, Wakayama Y, Osugi M, Hibi H. A defined mix of cytokines mimics conditioned medium from cultures of bone marrow-derived mesenchymal stem cells and elicits bone regeneration. Cell Prolif. 2017;50:e12333. https://doi.org/10.1111/cpr.12333
Gerwin N, Bendele AM, Glasson S, Carlson CS. The OARSI histopathology initiative—recommendations for histological assessments of osteoarthritis in the rat. Osteoarthritis Cartilage. 2010 Oct;18 Suppl 3:S24-34. doi: 10.1016/j joca.2010.05.030. PubMed PMID: 20864021.
Jacer, Sorayya, et al. “An Investigation on the Regenerative Effects of Intra Articular Injection of Co-Cultured Adipose Derived Stem Cells with Chondron for Treatment of Induced Osteoarthritis.” Advanced Pharmaceutical Bulletin, vol. 8, no. 2, 2018, pp. 297-306., doi:10.15171/apb.2018.035.
Tang, J. et al. Therapeutic microparticles functionalized with biomimetic cardiac stem cell membranes and secretome. Nat. Commun. 8, 13724 doi: 10.1038/ncomms13724 (2017).
U.S. Patent Application Publication No.: 20180361030
U.S. Patent Application Publication No.: 20180085318

1. A composition comprising one or more populations of microspheres, wherein each of the one or more populations of microspheres comprises at least one active agent.

2. The composition of claim 1, wherein the microspheres comprise a polymeric waxy or other protective material such as a natural, a semi-synthetic, or a synthetic polymer.

3. The composition of any of claims 1-2, wherein the microspheres have a diameter ranging from about 1 μm to about 100 μm.

4. The composition of any of claims 1-2, wherein the microspheres have a diameter ranging from about 10 μm to about 20 μm.

5. The composition of any of claims 1-4, wherein the microspheres comprise a biocompatible polymer selected from: Poly(lactic-co-glycolic acid) (PLGA), Poly(lactic acid) (PLA), Poly(E-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof.

6. The composition of any of claims 1-5, wherein the at least one active agent comprises one or more active agents selected from: growth factors, chemokines, cytokines, CD antigens, neurotrophins, and microRNAs (miRNAs).

7. The composition of any of claims 1-6, wherein the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMPS), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGFS), Fibroblast growth factor 6 (FGF6), Fibroblast growth factor 7(FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDFS), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth Factor, Kruppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1), Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOX5, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-(β), Transforming Growth Factor-β2 (TGF-β2), Transforming Growth Factor-β (TGF-(33), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor-D (VEGF-D).

8. The composition of any of claims 1-6, wherein the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCLS), Eotaxin (CCL11,24,26), Exodus-2 (CCL21), Fractalkine (CX3CL1), GRO (CXCL1,2,3), HCC-1 (CCL14), 1-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCLS), SDF (CXCL12), TARC (CCL17), CCL14, CCL19, CCL20, CCL27, CXCL13, and Thymus Expressed Chemokine (CCL25).

9. The composition of any of claims 1-6, wherein the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36γ, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, and Wingless-Type MMTV Integration Site Family.

10. The composition of any of claims 1-6, wherein the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CD5, CD8B, CD5L, CD68, CD55, CD7, CD73, CD58, CD74, CD80, CD79, CD84, CD93, CD99, CD164, and CD40L.

11. The composition of any of claims 1-6, wherein the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, and Pleiotrophin.

12. The composition of any of claims 1-6, wherein the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

13. The composition of any of claims 1-12, wherein the at least one active agent comprises VEGF, IGF-1, TGF-β, HGH, and FGF-18.

14. The composition of any of claims 1-12, wherein the at least one active agent comprises IGF-1, TGF-β, HGH, and FGF-18.

15. The composition of claim 14, wherein the composition comprises about 20% by weight FGF-18 microspheres, about 20% by weight TGF-β1 microspheres, about 20% by weight IGF-1 microspheres, about 20% by weight HGH microspheres, and about 20% by weight empty microspheres.

16. The composition of claim 14, wherein the composition comprises about 25% by weight FGF-18 microspheres, about 25% by weight TGF-β1 microspheres, about 25% by weight IGF-1 microspheres, and about 25% by weight HGH microspheres.

17. The composition of claim 14, wherein the composition comprises about 1 ng to about 100 ng HGH, about 100 ng HGH, about 90 ng HGH, about 80 ng HGH, about 70 HGH, about 60 ng HGH, about 50 ng HGH, about 40 ng HGG, about 30 ng HGH, about 20 ng HGH, about 10 ng HGH, about 9.256 ng HGH, about 9 ng HGH, about 8 ng HGH, about 7 ng HGH, about 6 ng HGH, about 5 ng HGH, about 4 ng HGH, about 3 ng HGH, about 2 ng HGH, or about 1 ng HGH.

18. The composition of claim 14, wherein the composition comprises about 1 ng to about 100 ng FGF-18, about 100 ng FGF-18, about 90 ng FGF-18, about 80 ng FGF-18, about 70 FGF-18, about 60 ng FGF-18, about 50 ng FGF-18, about 40 ng FGF-18, about 30 ng FGF-18, about 20 ng FGF-18, about 10 ng FGF-18, about 9.256 ng FGF-18, about 9 ng FGF-18, about 8 ng FGF-18, about 7 ng FGF-18, about 6 ng FGF-18, about 5 ng FGF-18, about 4 ng FGF-18, about 3 ng FGF-18, about 2 ng FGF-18, or about 1 ng FGF-18.

19. The composition of claim 14, wherein the composition comprises about 1 ng to about 100 ng IGF-1, about 100 ng IGF-1, about 90 ng IGF-1, about 80 ng IGF-1, about 70 IGF-1, about 60 ng IGF-1, about 50 ng IGF-1, about 40 ng IGF-1, about 30 ng IGF-1, about ng IGF-1, about 11.86 ng IGF-1, about 10 ng IGF-1, about 9 ng IGF-1, about 8 ng IGF-1, about 7 ng IGF-1, about 6 ng IGF-1, about 5 ng IGF-1, about 4 ng IGF-1, about 3 ng IGF-1, about 2 ng IGF-1, or about 1 ng IGF-1.

20. The composition of claim 14, wherein the composition comprises about 1 ng to about 100 ng TGF-β1, about 100 ng TGF-β1, about 90 ng TGF-β1, about 80 ng TGF-β1, about 70 TGF-β1, about 60 ng TGF-β1, about 50 ng TGF-β1, about 40 ng TGF-β1, about 30 ng TGF-β1, about 20 ng TGF-β1, about 10 ng TGF-β1, about 9.256 ng TGF-β1, about 9 ng TGF-β1, about 8 ng TGF-β1, about 7 ng TGF-β1, about 6 ng TGF-β1, about 5.424 ng TGF-β1, about 5 ng TGF-β1, about 4 ng TGF-β1, about 3 ng TGF-β1, about 2 ng TGF-β1, or about 1 ng TGF-β1.

21. The composition of any of claims 1-12, wherein the at least one active agent comprises VEGF, TGF-β1, and BMP2.

22. The composition of any of claims 1-12, wherein the at least one active agent comprises Myostatin, IGF-1, and Growth differentiation factor 11.

23. The composition of any of claims 1-12, wherein the at least one active agent comprises Oct-4, SOX2, KLF4, and c-Myc.

24. The composition of any of claims 1-23, wherein the biocompatible polymer comprises PLGA (Poly(lactic-co-glycolic acid)).

25. The composition of any of claims 13-24, wherein the microspheres have a diameter ranging from about 10 μm to about 20 μm.

26. The composition of any of claims 1-12, wherein the composition comprises two or more populations of microspheres and each population of microspheres comprises a single active agent.

27. The composition of any of claims 1-12, wherein the composition comprises one population of microspheres and the population of microspheres comprises two or more active agents.

28. The composition of any of claims 1-27, wherein the at least one active agent further comprises a carrier protein.

29. The composition of claims 28, wherein the carrier protein is selected from bovine serum albumin, human serum albumin, equine serum albumin, goat serum albumin, porcine serum albumin, rat serum albumin, mouse serum albumin, chicken serum albumin, and chicken white albumin.

30. The composition of any of claims 1-29, wherein the composition further comprises one or more delivery vehicles, diluents, excipients, pharmaceutical adjuvants, stimulants, and/or stabilizers.

31. A method for treating a subject, comprising administering an effective dose of the composition of any of claims 1-30.

32. The method of claim 31, wherein the subject is suspected of having or has a disease selected from the group consisting of: Alzheimer's disease, spinal cord injury, muscular dystrophy, osteoporosis, and osteoarthritis, and the method serves to treat Alzheimer's disease, spinal cord injury, muscular dystrophy, osteoporosis, and osteoarthritis.

33. A method for treating a spinal cord injury, muscular dystrophy, osteoporosis, and/or osteoarthritis in a subject, comprising administering an effective dose of the composition of any of claims 13-20.

34. A method for treating osteoarthritis (OA) in a subject, comprising administering an effective dose of the composition of any of claims 13-20.

35. A method for repairing bone defects in a subject, comprising administering an effective dose of the composition of claim 21.

36. A method for attenuating skeletal muscle degeneration in a subject, comprising administering an effective dose of the composition of claim 22.

37. A method for reprogramming cells in a subject, comprising administering an effective dose of the composition of claim 23.

38. A method for preparing a composition comprising microspheres and at least one active agent, the method comprising:
(a) dissolving a polymer in a polar solvent and mixing to prepare a dissolved polymer;
(b) mixing the at least one active agent with a buffer and a carrier protein to prepare a diluted active agent;
(c) combining the dissolved polymer with the diluted active agent and mixing to prepare a primary emulsion;
(d) mixing the primary emulsion with an aqueous surfactant to make a secondary emulsion;
(e) mixing the secondary emulsion in a bulk aqueous solution comprising a salt, the aqueous surfactant, and an alcohol until the polar solvent is evaporated; and
(f) isolating the microspheres.

39. The method of claim 38, wherein the polymer is selected from: Poly(lactic-co-glycolic acid) (PLGA), Poly(lactic acid) (PLA), Poly(ϵ-caprolactone) (PCL), Poly(glycolic acid) (PGA), Polyhydroxyalkonates (PHA), Polyphenylene ethylene (PPE), Polyphosphazenes, Poly(Methyl-Methacrylate) (PMMA), Poly D-lactic acid (PDLA), Poly(L-Lactic Acid) (PLLA), Poly(etherether ketone) (PEEK), Polyethylene glycol (PEG), Polyethylene glycol-diacrylate (PEGDA), Polyorthoester, Aliphatic polyanhydride, aromatic polyanhydrides, and/or block co-polymer thereof, and/or combinations thereof.

40. The method of either claim 38 or claim 39, wherein the polar solvent is selected from: Dichloromethane (DCM), Acetone, Acetonitrile, Chloroform, Dichloromethane (DCM), Dimethyl Sulfoxide (DMSO), Dimethyl Carbonate (DMC), Dimethylacetamide (DMAc), Dimethylformamide (DMF), Ethyl Acetate, Methanol, N-Methyl-2-Pyrrolidone (NMP), and Tetrahydrofuran (THF).

41. The method of any of claims 38-40, wherein the at least one active agent comprises one or more active agents selected from: growth factors, chemokines, cytokines, CD antigens, neurotrophins, and microRNAs.

42. The method of any of claims 38-41, wherein the at least one active agent comprises one or more growth factors selected from: Activin, Bone Morphogenic protein (BMP), Bone Morphogenic protein 1 (BMP1), Bone Morphogenic protein 2 (BMP2), Bone Morphogenic protein 3 (BMP3), Bone Morphogenic protein 4 (BMP4), Bone Morphogenic protein 5 (BMPS), Bone Morphogenic protein 6 (BMP6), Bone Morphogenic protein 7 (BMP7), Bone Morphogenic protein 8a (BMP8a), Bone Morphogenic protein 8b (BMP8b), Bone Morphogenic protein 10 (BMP10), Bone Morphogenic protein 11 (BMP11), Bone Morphogenic protein 15 (BMP15), Colony-Stimulating Factor 1 (CSF1), Colony-Stimulating Factor 2 (CSF2), Colony-Stimulating Factor 3 (CSF3), Connective Tissue Growth Factor (CTGF), Epidermal Growth-Factor (EGF), Epigen, Erythropoietin, Heparin-binding EGF-like growth factor (HB-EGF), Amphiregulin (AR), Epiregulin (EPR), Betacellulin (BTC), neuregulin-1 (NRG1), neuregulin-2 (NRG2), neuregulin-3 (NRG3), neuregulin-4 (NRG4), Fibroblast Growth Factor (FGF), Fibroblast growth factor 1 (FGF1), Fibroblast growth factor 2 (FGF2), Fibroblast growth factor 3 (FGF3), Fibroblast growth factor 4 (FGF4), Fibroblast growth factor 5 (FGFS), Fibroblast growth factor 6 (FGF6), Fibroblast growth factor 7 (FGF7), Fibroblast growth factor 8 (FGF8), Fibroblast growth factor 9 (FGF9), Fibroblast growth factor 10 (FGF10), Fibroblast growth factor 11 (FGF11), Fibroblast growth factor 12 (FGF12), Fibroblast growth factor 13 (FGF13), Fibroblast growth factor 14 (FGF14), Fibroblast growth factor 15 (FGF15), Fibroblast growth factor 16 (FGF16), Fibroblast growth factor 17 (FGF17), Fibroblast growth factor 18 (FGF18), Fibroblast growth factor 19 (FGF19), Fibroblast growth factor 20 (FGF20), Fibroblast growth factor 21 (FGF21), Fibroblast growth factor 22 (FGF22), Fibroblast growth factor 23 (FGF23), Galectin, Granulocyte-Macrophage Colony-Stimulating Factor (GM-CSF), Growth Differentiation Factor (GDF), Growth Differentiation Factor 1 (GDF1), Growth Differentiation Factor 2 (GDF2), Growth Differentiation Factor 3 (GDF3), Growth Differentiation Factor 5 (GDF5), Growth Differentiation Factor 6 (GDF6), Growth Differentiation Factor 8 (GDF8), Growth Differentiation Factor 9 (GDF9), Growth Differentiation Factor 10 (GDF10), Growth Differentiation Factor 11 (GDF11), Growth Differentiation Factor 15 (GDF15), Human Growth Hormone (HGH), Hepatoma-Derived Growth Factor (HDGF), Hepatocyte Growth Factor (HGF), Insulin-Like Growth Factor Binding Protein (IGFBP), Insulin-Like Growth Factor-1 (IGF-1), Insulin-Like Growth Factor-2 (IGF-2), Insulin, Keratinocyte Growth Factor, Kruppel-like family of transcription factor proteins (KLF; KLF1, KLF2, KLF3, KLF4, KLF5, KLF6, KLF7, KLF8, KLF9, KLF10, KLF11, KLF12, KLF13, KLF14, KLF15, KLF16, or KLF17) Leptin, Macrophage Migration Inhibitory Factor, Melanoma Inhibitory Activity, MYC proto-oncogene (MYC proto-oncogene bHLH transcription factor; c-Myc), Myostatin, Noggin, Nephroblastoma-overexpressed (NOV), Nerve Growth Factor (NGF), Octamer transcription factor proteins (OCT), Oct-1 (POU2F1), Oct-2 (POU2F2), Oct-3/4 (POU5F1), Oct-6 (POU3F1), Oct-7 (POU3F2), Oct-8 (POU3F3), Oct-9 (POU3F4), Oct-11 (POU3F4), Omentin, Oncostatin-M, Osteopontin, Osteoprotegerin, Platelet-Derived Growth Factor (PDGF), Periostin, Placental Growth Factor 1 (PGF1), Placental Growth Factor 2 (PGF2), Placental Growth Factor 3 (PGF3), Placental Lactogen, Prolactin (PRL), RANK Ligand, Retinol Binding Protein, SRY-related HMG-box proteins (SOX proteins), SoxA (SRY), SoxB1 (SOX1, SOX2, SOX3), SoxB2 (SOX14, SOX21), SoxC (SOX4, SOX11, SOX12), SoxD (SOX5, SOX6, SOX13), SoxE (SOX8, SOX9, SOX10), SoxF (SOX7, SOX17, SOX18), SoxG (SOX15), SoxH (SOX30), Stem Cell Factor (SCF), Transforming Growth Factor-α (TGF-α), Transforming Growth Factor-β (TGF-β), Transforming Growth Factor-β2 (TGF-β2), Transforming Growth Factor-β3 (TGF-β3), Vascular Endothelial Growth Factor (VEGF), Vascular Endothelial Growth Factor-A (VEGF-A), Vascular Endothelial Growth Factor-B (VEGF-B), Vascular Endothelial Growth Factor-C (VEGF-C), and Vascular Endothelial Growth Factor-D (VEGF-D).

43. The method of any of claims 38-41, wherein the at least one active agent comprises one or more chemokines selected from: BCA-1/BLC (CXCL13), BRAK (CXCL14), C-10 (CCL6), CTACK (CCL27), CXCL16, CXCL17, CXCL6, ENA-78 (CXCL5), Eotaxin (CCL11,24,26), Exodus-2 (CCL21), Fractalkine (CX3CL1), GRO (CXCL1,2,3), HCC-1 (CCL14), 1-309 (CCL1), Interleukin 8 (CXCL8), IP-10 (CXCL10), I-TAC (CXCL11), LD78-beta (CCL3L1), Lymphotactin (XCL1), MCP (CCL2, 7,8,12,13), MDC (CCL22), MEC (CCL28), MIG (CXCL9), MIP (CCL3,4,9,15), NAP-2 (CXCL7), Platelet Factor-4 (CXCL4), Rantes (CCL5), SDF (CXCL12), TARC (CCL17), CCL14, CCL19, CCL20, CCL27, CXCL13, and Thymus Expressed Chemokine (CCL25).

44. The method of any of claims 38-41, wherein the at least one active agent comprises one or more cytokines selected from: 4-1BB, Adiponectin, AITRL, AIF1, Angiopoietin, Apolipoprotein, B-Cell Activating Factor, Beta Defensin, Betacellulin, Bone Morphogenetic Protein, BST, B type Natriuretic Peptide, Cardiotrophin, CTLA4, EBI3, Endoglin, Epiregulin, FAS, Flt3 Ligand, Follistatin, Hedgehog Protein, Interferon, Interleukin (IL), IL-1α, IL-1β, IL-1ra, IL-18, IL-33, IL-36α, IL-36β, IL-36γ, IL-36ra, IL-37, IL-38, Leukemia Inhibitory Factor, Otoraplin, Resistin, Serum Amyloid A, TPO, Trefoil Factor, TSLP, Tumor Necrosis Factor, Uteroglobin, Visfatin, and Wingless-Type MMTV Integration Site Family.

45. The method of any of claims 38-41, wherein the at least one active agent comprises one or more CD antigens selected from: CD1, CD14, CD2, CD200, CD204, CD207, CD226, CD244, CD27, CD23, CD274, CD247, CD3, CD33, CD300, CD34, CD36, CD4, CD40, CD46, CD47, CDS, CD8B, CDSL, CD68, CD55, CD7, CD73, CD58, CD74, CD80, CD79, CD84, CD93, CD99, CD164, and CD40L.

46. The method of any of claims 38-41, wherein the at least one active agent comprises one or more neurotrophins selected from: BDNF, Beta-NGF, CDNF, CNTF, GDNF, Glia Maturation Factor, MANF, Midkine, Neuregulin, Neuroglobin, Neuritin, Neuropilin, Neurotrophic factor, Persephin, Pigment Epithelium-Derived Factor, and Pleiotrophin.

47. The method of any of claims 38-41, wherein the at least one active agent comprises one or more microRNAs selected from: miRNA-1, miRNA-140, miRNA-204, miRNA-211, miRNA-9, miRNA-31, miRNA-124, miRNA-124, miRNA-146a, miRNA-365, miRNA-133, miRNA-206, and miRNA-499.

48. The method of any of claims 38-47, wherein the buffer is selected from: Acetate buffers, ACES, ADA, AMP, AMPSO, AMPD, BES, Bicine, Bis-Tris, Bis-Tris Propane, CABS, CAPSO, CAPS, CHES, Citrate buffers, DIPSO, EPPS, Gly-Gly, HEPBS, HEPES, HEPPSO, PBS, PIPES, POPSO, MES, MOBS, MOPSO, MOPS, Sodium carbonate buffers, Sodium bicarbonate buffers, TABS, TAPS, TAPSO, TBS, TEA, TES, Tricine, and Tris.

49. The method of any of claims 38-48, wherein the carrier protein is selected from bovine serum albumin, human serum albumin, equine serum albumin, goat serum albumin, porcine serum albumin, rat serum albumin, mouse serum albumin, chicken serum albumin, and chicken white albumin.

50. The method of any of claims 38-49, wherein the salt of the bulk aqueous solution is selected from: chloride salts (e.g., NaCl), fluoride salts, phosphate salts, iron salts, carbonate salts, bicarbonate salts, sulfate salts, bisulfate salts, and potassium dichromate.

51. The method of any of claims 38-50, wherein the aqueous surfactant of the bulk aqueous solution is selected from: poly vinyl alcohol (PVA), polysorbate (e.g. Tweens), sorbitan esters (SPAN), poly(vinyl pyrrolidone) (PVP; also known as povidone, and poloxamers).

52. The method of any of claims 38-51, wherein the alcohol of the bulk aqueous solution is selected from: ethanol, propanol, n-butanol, 1-pentanol, and structural and functional isomers thereof.

53. The method of any of claims 38-52, further comprising lyophilizing the isolated microspheres.

54. The method of any of claims 38-53, further comprising sieving the isolated microspheres and collecting the microspheres that are between about 10 μm to about 20 μm in diameter.

55. The method of any of claims 38-54, wherein the ratio of the polar solvent to the aqueous surfactant of the secondary emulsion is about 1:20 to about 1:1.
